SEQUENCE LISTING (2 sequences)

SEQ ID NO: 1 - protein, 8 aa, Homo sapiens [sequence omitted]

SEQ ID NO: 2 - protein, 7 aa, Homo sapiens
YMGWMDF

(19) United States
(12) Patent Application Publication
(10) Pub. No.: US 2009/0281032 A1
(43) Pub. Date: Nov. 12, 2009

(54) Modified CCK peptides

(76) Inventors: Peter Raymond Flatt, Portrush (GB); Finbarr Paul Mary O'Harte, Portstewart (GB)

Correspondence Address: Licata & Tyrrell P.C., 66 E. Main Street, Marlton, NJ, 08053, US

(21) Appl. No.: 12/177,306

(22) Filed: Jul. 22, 2008

Related U.S. Application Data
(63) Continuation-in-part of application No. 10/469,655, filed on Feb. 5, 2004, now Pat. No. 7,402,663.
Application No. 10/469,655 is a continuation of application No. PCT/GB02/00827, filed on Feb. 28, 2002.

(30) Foreign Application Priority Data
Mar. 1, 2001 (GB) 0105069.9

(51) Int. Cl. A61K 38/17; A61P 3/10; C07K 1/06; C07K 7/06; A61K 38/10

(52) U.S. Cl. 514/12; 530/334; 530/328; 530/329; 514/14

(57) ABSTRACT

The invention concerns a peptide based on biologically active CCK-8. The peptide has improved characteristics for the treatment of at least one of obesity and type 2 diabetes and has the structure:

(Z)-Asp¹-Aaa²(X)-Aaa³Gly⁴Trp⁵Aaa⁶Asp⁷(Y)Aaa⁸K,

wherein the amino acids may be either D or L amino acids; the bond between amino acid residues is either a peptide bond or a non-peptide isostere bond; Aaa² is selected from the group comprising Tyr and Phe; when Aaa² is Tyr, X is selected from the group comprising SO₃H⁻, PO₃H₂⁻ and a polymer moiety of the general formula —O—(CH₂—O—CH₂)ₙ—H, in which n is an integer between 1 and about 22, wherein the X is covalently bound to the para phenyl oxygen of Tyr, and, when Aaa² is Phe, X is CH₂SO₃Na, wherein the X is covalently bound to the para phenyl position of Phe; Aaa³ is selected from the group comprising Met, norleucine, 2-aminohexanoic acid and Thr; Aaa⁶ is selected from the group comprising Met, norleucine, 2-aminohexanoic acid and Phe; Aaa⁸ is selected from the group comprising Phe and Met; Y is covalently bound to the nitrogen of Aaa⁸ and is selected from the group consisting of H and CH₃; K is selected from the group consisting of the hydroxyl group of Phe⁸, an amide covalently bound to Phe⁸, an ester covalently bound to Phe⁸, a salt of the hydroxyl group of Phe⁸, a salt of an amide covalently bound to Phe⁸, a salt of an ester covalently bound to Phe⁸ and a polymer moiety of the general formula —O—(CH₂—O—CH₂)ₙ—H, in which n is an integer between 1 and about 22; and Z comprises at least one amino acid modification, wherein said at least one modification comprises an N-terminal extension, or an N-terminal modification, but excludes Asp¹-glucitol CCK-8 where Aaa² is Tyr and X is SO₃H⁻.

The peptides, and Asp¹-glucitol CCK-8, are useful to at least one of inhibit food intake, induce satiety, stimulate insulin secretion, moderate blood glucose excursions, enhance glucose disposal and exhibit enhanced stability in plasma compared to native CCK-8

The present invention relates to the regulation of feeding and control of energy metabolism. More particularly the invention relates to the use of peptides to suppress food intake and pharmaceutical preparations for the treatment of obesity and type 2 diabetes.

Obesity and type 2 diabetes are two of the most common metabolic disorders in western societies. The risks to health posed by obesity are considerable, including predisposition to diabetes and its associated long-term complications. Despite this worldwide epidemic, there is currently only a limited number of drugs available to counter these major metabolic diseases. These are largely ineffective in the case of obesity or unable to prevent development of complications in diabetes.

The present invention concerns the discovery of novel modified long-acting analogues of CCK-8 and their potential use for regulation of appetite control and treatment of obesity and related diabetes. The insulin-releasing capability of these analogues is also directly beneficial in terms of improved blood glucose control, thereby making these agents a novel class of antidiabetic agent.

The regulation of food intake is a complex process that is controlled by a system of hunger and satiety signals interacting in complex pathways both peripherally and centrally (Ukkola 2004). Signals from the gastrointestinal tract, pancreas and adipose tissue together with circulating nutrients converge on the hypothalamus to regulate food intake and energy expenditure. The arcuate nucleus (ARC), in particular, is thought to play a pivotal role in the integration of these signals (Wynne et al. 2005). A growing number of peptides have been discovered which elicit the ability to decrease food intake (anorexigenic peptides) or increase food intake (orexigenic peptides) in animals and humans. As a group, they provide a number of leads for potential drug development.

Cholecystokinin (CCK) is a neuropeptide hormone found in the brain and secreted from gut endocrine cells, which was originally identified from its ability to stimulate gall bladder contraction. CCK is now known to play a significant role in many physiological processes including regulation of satiety, bowel motility, gastric emptying, insulin secretion, pancreatic enzyme secretion and neurotransmission. Cholecystokinin is a neuropeptide hormone released postprandially by gut endocrine I cells (Liddle 1994). CCK-8 acts via two major receptor sub-populations CCKA (peripheral) and CCKB (brain) (Innis et al. 1980). CCK exists in multiple molecular forms in the circulation ranging from 58, 39, 33, 22, 8 and 4 amino acids in length (Cantor 1989, Inui 2000). CCK-33 was the original form purified from porcine intestine. The C-terminal octapeptide CCK-8 is well conserved between species and is the smallest form that retains the full range of biological activities (Smith 1984, Crawley & Corwin 1995, Inui 2000). A variety of CCK molecular forms are secreted following ingestion of dietary fat and protein, from endocrine mucosal I cells that are mainly located in the duodenum and proximal jejunum. Once released, CCK-8 exerts its biological action on various target tissues within the body in a neurocrine, paracrine or endocrine manner. These actions are mediated through two major receptor sub-populations CCK_{A} (peripheral subtype) and CCK_{B} (brain subtype). Specific receptor antagonists such as proglumide have aided our understanding of the action of CCK on food intake.

CCK receptors are also present in pancreatic islets. CCK-8 has been shown to reduce feeding dose dependently in a variety of species including man (Gibbs et al. 1973, Morley 1987, Silver et al. 1991). Involvement of CCK in the control of food intake in rodents was recognised in the early 1970's, and since then this peptide hormone has been shown to reduce feeding in man and in several animal species. The induction of satiety is a common feature in different species but the mechanism by which this is achieved is poorly understood. However, many different tissues are known to possess specific receptors for CCK including the vagus nerve, pyloric sphincter and brain, all of which may be implicated in this satiety control mechanism. It has been proposed that CCK stimulates receptors in the intestine that activate the vagus nerve, which relays a message to the satiety centres in the hypothalamus. In support of this concept, it has been found that satiety effects of CCK are eliminated in vagotomized animals. Furthermore, rodent studies have indicated that CCK has a more potent satiating ability when administered by the intraperitoneal route rather than centrally. Intraperitoneal CCK-8 is thought to act locally rather than hormonally. In addition, it is known that CCK-8 does not cross the blood brain barrier.

Nevertheless, other evidence suggests that CCK has a definite neuronal influence on food intake in the central nervous system. Some work in dogs has suggested that circulating levels of CCK were too low to induce satiety effects. However, studies in pigs immunized against CCK revealed that these animals increased their food intake and had accelerated weight gain compared to control animals. In addition CCK receptor antagonists increased food intake in pigs and decreased satiety in humans. Overall the above studies indicate that CCK plays a significant role in regulating food intake in mammals.

CCK-8 has been considered as a short-term meal-related satiety signal whereas the recently discovered OB gene product leptin, is more likely to act as an adiposity signal which may reduce total food intake over the longer term. Indeed some workers have suggested that CCK-8 and leptin act synergistically to control long term feeding in mice.

The present invention aims to provide effective analogues of CCK-8. It is one aim of the invention to provide pharmaceuticals for treatment of obesity and/or type 2 diabetes.

According to the present invention there is provided an effective peptide analogue of the biologically active CCK-8 which has improved characteristics for the treatment of obesity and/or type 2 diabetes wherein the analogue has at least one amino acid substitution or modification and not including Asp¹-glucitol CCK-8.

The primary structure of human CCK-8 is shown below:

Asp¹Tyr²(SO₃H)-Met³Gly⁴Trp⁵Met⁶Asp⁷Phe⁸ amide

The analogue may include modification by fatty acid addition (e.g., palmitoyl) at the alpha amino group of Asp¹ or an epsilon amino group of a substituted lysine residue. The invention includes Asp¹-glucitol CCK-8 having fatty acid addition at an epsilon amino group of at least one substituted lysine residue.

By glucitol is meant

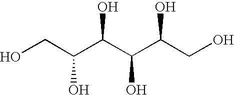

and by Asp¹-glucitol is meant the moiety in which a hydroxyl group of glucitol is reacted with the amino group of an amino acid.

Analogues of CCK-8 may have an enhanced capacity to inhibit food intake, stimulate insulin secretion, enhance glucose disposal or may exhibit enhanced stability in plasma compared to native CCK-8. They may also possess enhanced resistance to degradation by naturally occurring exo- and endo-peptidases.

Any of these properties will enhance the potency of the analogue as a therapeutic agent.

Analogues having one or more D-amino acid substitutions within CCK-8 and/or N-glycated, N-alkylated, N-acetylated, N-acylated, N-isopropyl, N-pyroglutamyl, pGluGln amino acids at position 1 are included.

Analogues having one or more D-amino acid substitutions within CCK-8 and/or N-glycated, N-alkylated, N-acetylated, N-acylated, N-isopropyl, N-pyroglutamyl amino acids at position 1 are included.

By pyroglutamic acid is meant:

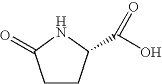

and by pyroglutamyl is meant the moiety in which the hydroxyl group of the carboxyl group of pyroglutamic acid is reacted with the amino group of another amino acid.

Various amino acid substitutions including for example, replacement of Met³ and/or Met⁶ by norleucine or 2-aminohexanoic acid. Various other substitutions of one or more amino acids by alternative amino acids include replacing Met³ by Thr, Met⁶ by Phe, Phe⁸ by N-methyl Phe.

Other stabilised analogues include those with a peptide isostere bond replacing the normal peptide bond between residues 1 and 2 as well as at any other site within the molecule. Furthermore, more than one isostere bond may be present in the same analogue. These various analogues should be resistant to plasma enzymes responsible for degradation and inactivation of CCK-8 in vivo, including for example aminopeptidase A.

In particular embodiments, the invention provides a peptide which is more potent than CCK-8 in inducing satiety, inhibiting food intake or moderating blood glucose excursions, said peptide consisting of CCK(1-8), or smaller fragment, with one or more modifications selected from the group consisting of:

(i) N-terminal extension of CCK-8 by pGlu-Gln;
(ii) N-terminal extension of CCK-8 by pGlu-Gln with substitution of Met⁸ by Phe;
(iii) N-terminal extension of CCK-8 by Arg;
(iv) N-terminal extension of CCK-8 by pyroglutamyl (pGlu);
(v) substitution of the penultimate Tyr² (SO₃H) by a phosphorylated Tyr;
(vi) substitution of the penultimate Tyr² (SO₃H) by Phe(pCH₂SO₃Na);
(vii) substitution of a naturally occurring amino acid by an alternative amino acid including; Met³ and/or Met⁶ by norleucine or 2-aminohexanoic acid, Met³ by Thr, Met⁶ by Phe, Phe⁸ by N-methyl Phe;
(viii) substitution described in (vii) above with or without N-terminal modification of Asp¹ (e.g., by acetylation, glycation, acylation, alkylation, isopropylation, pGlu, pGlu-Gln etc);
(ix) modification of Asp¹ by acetylation;
(x) modification of Asp¹ by acylation (e.g., palmitate);
(xi) modification of a substituted Lys residue by a fatty acid (e.g., palmitate);
(xii) modification of Asp¹ by alkylation;
(xiii) modification of Asp¹ by glycation in addition to a fatty acid (e.g., palmitate) linked to an epsilon amino group of a substituted Lys residue;
(xiv) modification of Asp¹ by isopropyl;
(xv) modification of Asp¹ by Fmoc or Boc;
(xvi) conversion of Asp¹-Tyr² bond to a stable non-peptide isostere bond CH₂NH;
(xvii) conversion of Tyr²-Met³ bond to a psi [CH₂NH] bond;
(xviii) conversion of Met³-Gly⁴ bond to a psi [CH₂NH] bond;
(xix) conversion of Met⁶-Asp⁷ bond to a psi [CH₂NH] bond;
(xx) conversion of other peptide bonds to a psi [CH₂NH] bond;
(xxi) modification of Tyr² by acetylation (i.e. acetylated CCK-7);
(xxii) modification of Tyr² by pyroglutamyl (i.e. pyroglutamyl CCK-7);
(xxiii) modification of Tyr² by glycation (i.e. glycated CCK-7);
(xxiv) modification of Tyr² by succinic acid (i.e. succinyl CCK-7);
(xxv) modification of Tyr² by Fmoc (i.e. Fmoc CCK-7);
(xxvi) modification of Tyr² by Boc (i.e. Boc CCK-7);
(xxvii) D-amino acid substituted CCK-8 at one or more sites;
(xxviii) D-amino acid substituted CCK-8 at one or more sites in addition to an N-terminal modification by, for example, acetylation, acylation, glycation, alkylation, isopropylation, pGlu, pGluGln, etc;
(xxix) reteroinverso CCK-8 (substituted by D-amino acids throughout octapeptide and primary structure synthesised in reverse order); and
(xxx) shortened N- and/or C-terminal truncated forms of CCK-8 and cyclic forms of CCK-8.

The invention also provides a method of N-terminally modifying CCK-8, or analogues thereof, during synthesis. Preferably, the agents would be glucose, acetic anhydride or pyroglutamic acid.

The invention also provides the use of Asp¹-glucitol CCK-8, pGlu-Gln CCK-8 and other analogues in the preparation of medicament for treatment of obesity and/or type 2 diabetes.

The invention further provides improved pharmaceutical compositions including analogues of CCK-8 with improved pharmacological properties.

Other possible analogues include truncated forms of CCK-8 represented by removal of single or multiple amino acids from either the C- or N-terminus in combination with one or more of the other modifications specified above.

According to the present invention there is also provided a pharmaceutical composition useful in the treatment of obesity and/or type 2 diabetes which comprises an effective amount of the peptide as described herein, in admixture with a pharmaceutically acceptable excipient for delivery through transdermal, nasal inhalation, oral or injected routes. Said peptide can be administered alone or in combination therapy with native or derived analogues of leptin, exendin, islet amyloid polypeptide (IAPP) or bombesin (gastrin-releasing peptide).

The invention also provides a method of N-terminally modifying CCK-8 and analogues thereof. This 3 step process firstly involves solid phase synthesis of the C-terminus up to Met³. Secondly, adding Tyr(tBu) to a manual bubbler system as an Fmoc-protected PAM resin, deprotecting the Fmoc by piperidine in DMF and reacting with an Fmoc protected Asp(OtBu)-OH, allowing the reaction to proceed to completion, removal of the Fmoc protecting group from the dipeptide, reacting the dipeptide with the modifying agent (e.g., glucose, acetic anhydride, palmitate, etc), removal of side-chain protecting groups (tBu and OtBu) by acid, sulphating the Tyr² with sulphur trioxide, cleaving the peptide from the resin under alkaline conditions. Thirdly, the N-terminal modified dipeptide can be added to the C-terminal peptide resin in the synthesizer, followed by cleavage from the resin under alkaline conditions with methanolic ammonia, and finally purification of the final product using standard procedures.

### SUMMARY OF THE INVENTION

According to a first aspect of the present invention there is provided a peptide based on biologically active CCK-8, the peptide having improved characteristics for the treatment of at least one of obesity and type 2 diabetes, wherein the structure of the peptide is:

(Z)-Asp¹-Aaa²(X)-Aaa³Gly⁴Trp⁵Aaa⁶Asp⁷(Y)Aaa⁸K,

wherein:
the amino acids may be either D or L amino acids;
the bond between amino acid residues is either a peptide bond or a non-peptide isostere bond;
Aaa² is selected from the group comprising Tyr and Phe;
when Aaa² is Tyr, X is selected from the group comprising SO₃H⁻, PO₃H₂⁻ and a polymer moiety of the general formula HO—(CH₂—O—CH₂)ₙ—H, in which n is an integer between 1 and about 22, wherein the X is covalently bound to the para phenyl oxygen of Tyr, and, when Aaa² is Phe, X is CH₂SO₃Na, wherein the X is covalently bound to the para phenyl position of Phe;
Aaa³ is selected from the group comprising Met, norleucine, 2-aminohexanoic acid and Thr;
Aaa⁶ is selected from the group comprising Met, norleucine, 2-aminohexanoic acid and Phe;
Aaa⁸ is selected from the group comprising Phe and Met;
(Y)Aaa⁸K, when Aaa⁸ is Phe⁸ and K is an amide, is:

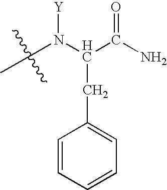

Y is covalently bound to nitrogen and is selected from the group consisting of H and CH₃;
K is selected from the group consisting of the hydroxyl group of Phe⁸, an amide covalently bound to Phe⁸, an ester covalently bound to Phe⁸, a salt of the hydroxyl group of Phe⁸, a salt of an amide covalently bound to Phe⁸, a salt of an ester covalently bound to Phe⁸, and a polymer moiety of the general formula —O—(Cl₂—O—CH₂)ₙ—H, in which n is an integer between 1 and about 22; and
Z comprises at least one amino acid modification, wherein said at least one modification comprises an N-terminal extension, or an N-terminal modification, but excludes Asp¹-glucitol CCK-8 where Aaa² is Tyr and X is SO₃H⁻.

Optionally, the structure of the peptide is:

(Z)-Asp¹-Aaa²(X)-Aaa³Gly⁴Trp⁵Aaa⁶Asp⁷(Y)Aaa⁸K,
wherein:
   the amino acids are L amino acids;
   the bonds between amino acid residues are peptide bonds;
   Aaa³ and Aaa⁶ are each Met;
   Aaa⁸ is Phe;
   Aaa²(X) is Tyr²(X) being:

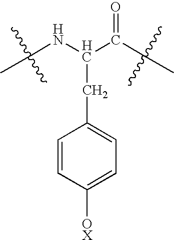

X is covalently bound to oxygen and selected from the group consisting of SO₃H⁻, PO₃H₂⁻ and a polymer moiety of the general formula —O—(CH₂—O—CH₂)ₙ—H, in which n is an integer between 1 and about 22;
K is an amide covalently bound to Phe⁸; and
Y is selected from the group consisting of H and CH₃.

Further optionally, said N-terminal modification at position 1 is selected from the group comprising N-alkylation, N-acetylation, N-acylation, N-glycation and N-isopropylation of the amino acid at position 1. Still further optionally, said N-terminal extension is selected from the group comprising pGlu, pGlu-Gln, an acid, a fatty acid, Boc, Fmoc, Arg and attachment of a polymer moiety of the general formula HO—(CH₂—O—CH₂)ₙ—H, in which n is an integer between 1 and about 22.

Optionally, the peptide is further modified by replacement of any amino acid with Lys, with or without fatty acid addition at an epsilon amino group of at least one substituted lysine residue.

Optionally, the peptide is further modified by attachment to Asp⁷ of a polymer moiety of the general formula HO—(CH₂—O—CH₂)ₙ—H, in which n is an integer between 1 and about 22. Further optionally, the peptide is modified by replacement of any amino acid with an amino acid selected from the group including, but not limited to, lysine, cysteine, histidine, arginine, aspartic acid, glutamic acid, serine, threonine, and tyrosine and attachment of a polymer moiety of the general formula HO—(CH₂—O—CH₂)ₙ—H, in which n is an integer between 1 and about 22 to at least one substituted amino acid.

Optionally, Z is selected from the group consisting of:

(i) N-terminal extension of the peptide by pGlu-Gln and Aaa⁸ is Phe;
(ii) N-terminal extension of the peptide by pGlu-Gln and Aaa⁸ is Met;
(iii) N-terminal extension of the peptide by Arg;
(iv) N-terminal extension of the peptide by pyroglutamyl (pGlu);
(v) modification of Asp¹ by acetylation;
(vi) modification of Asp¹ by acylation;
(vii) modification of Asp¹ by alkylation or glycation;
(viii) modification of Asp¹ by isopropylation;
(ix) N-terminal extension of the peptide at Asp¹ by Fmoc or Boc;
(x) N-terminal extension of the peptide by attachment of a polymer moiety of the general formula HO—(CH₂—O—CH₂)ₙ—H, in which n is an integer between 1 and about 22; and
(xi) N-terminal extension of the peptide by pGlu-Gln and C-terminal extension of the peptide by attachment of a polymer moiety of the general formula HO—(CH₂—O—CH₂)ₙ—H, in which n is an integer between 1 and about 22.

Alternatively or additionally, the peptide is modified by

(i) D-amino acid substituted CCK-8 at one or more amino acid sites and Z comprises an N-terminal extension or an N-terminal modification;
(ii) reteroinverso CCK-8 (substituted by D-amino acids throughout octapeptide and primary structure synthesised in reverse order); and
(iii) X is PO₃H₂⁻.

Optionally, at least one of K, X and Z comprises a polymer moiety covalently bound to Phe⁸, the polymer moiety being of the general formula HO—(CH₂—O—CH₂)ₙ—H, in which n is an integer between 1 and about 22; further optionally, wherein n is an integer between 1 and about 10; still further optionally, wherein n is an integer between about 2 and about 6.

Optionally, when K comprises a polymer moiety covalently bound to Phe⁸, the polymer moiety is of the general formula —O—(CH₂—O—CH₂)ₙ—H, in which n is an integer between 1 and about 22, the peptide is further modified by N-terminal extension of the peptide, wherein the peptide is, optionally, modified by N-terminal extension of the peptide by pGlu-Gln.

Optionally, in the peptide of the present invention,
the amino acids are L amino acids;
the bonds between amino acid residues are peptide bonds;
Aaa³ and Aaa⁶ are each Met;
Aaa⁸ is Phe;
Aaa² is Tyr²;
X is PO₃H₂⁻;
K is an amide covalently bound to Phe⁸; and
Y is selected from the group consisting of H and CH₃.

Optionally, in the peptide of the present invention,
the amino acids are L amino acids;
the bonds between amino acid residues are peptide bonds;
Aaa³ and Aaa⁶ are each Met;
Aaa⁸ is Phe;
Aaa² is Tyr²;
X is SO₃H⁻;
K is an amide covalently bound to Phe⁸;
Y is selected from the group consisting of H and CH₃; and
   the peptide is modified by N-terminal acetylation of Asp¹.

Optionally, there is at least one peptide isostere bond is present between amino acid residues at any site within the peptide. For example, an isostere bond may be present between Asp¹-Tyr²; between Tyr²-Met³; between Met³-Gly⁴; or between Met⁶-Asp⁷.

Optionally, Z is selected from the group consisting of:

(i) N-terminal extension of the peptide by pGlu-Gln;
(ii) N-terminal extension of the peptide by Arg;
(iii) N-terminal extension of the peptide by pyroglutamyl (pGlu);
(iv) modification of Asp¹ by acetylation;
(v) modification of Asp¹ by acylation;
(vi) modification of Asp¹ by alkylation or glycation;
(vii) modification of Asp¹ by isopropylation; and

The invention further provides an effective peptide analogue of the biologically active CCK-8 which has improved characteristics for the treatment of obesity and/or type 2 diabetes, wherein the analogue has at least one amino acid substitution or modification, wherein said at least one amino acid substitution or modification comprises attachment of a polymer moiety to the CCK-8 analogue, or peptide fragment, of the general formula —O—(CH₂—O—CH₂)ₙ—H, in which n is an integer between 1 and about 22. Optionally, the polymer moiety has an average molecular weight of no more than 1000 Da. Preferably, the polymer moiety has an average molecular weight of less than 1000 Da. Preferably, n is an integer between 1 and about 10. More preferably, n is an integer between about 2 and about 6. Optionally, the polymer molecule has a branched structure. The branched structure may comprise the attachment of at least two polymer moieties of linear structure. Alternatively, the branch point may be located within the structure of each polymer moiety. Alternatively, the polymer moiety has a linear structure. Some or all monomers of the polymer moiety can be associated with water molecules. Attachment of the polymer moiety can be achieved via a covalent bond. Optionally, the covalent bond is a stable covalent bond. Alternatively, the covalent bond is reversible. The covalent bond can be hydrolysable. The or each polymer moiety can be attached adjacent the N-terminal amino acid; adjacent the C-terminal amino acid; or to a naturally occurring amino acid selected from the group including, but not limited to, aspartic acid and tyrosine. Alternatively, the peptide analogue further comprises substitution of a naturally occurring amino acid with an amino acid selected from the group including, but not limited to, lysine, cysteine, histidine, arginine, aspartic acid, glutamic acid, serine, threonine, and tyrosine; the or each polymer moiety being attached to the or each substituted amino acid. Optionally, the or each polymer moiety is attached adjacent the C-terminal amino acid. Further optionally, the or each polymer moiety is attached to the C-terminal amino acid.

Polyethylene glycol (PEG) is a polymer having the general structure: HO—(CH₂—CH₂—O)ₙ—H, which is produced from the polymerisation of ethylene glycol monomers (C₂H₄(OH)₂), by the interaction of ethylene oxide with water, ethylene glycol or ethylene glycol oligomers, the reaction being catalysed by acidic or basic catalysts. Polymer chain length is determined by the number of ethylene glycol monomers (n), and is dependent on the ratio of reactants.

The covalent attachment of one or more polyethylene glycol molecules (PEGs) to CCK-8 analogues, or peptide fragments, has been investigated with the goal of improving the pharmacokinetic behaviour of therapeutic drugs. The use of peptide-based therapeutic agents, in particular, is hampered by several disadvantages. Primarily, the peptide is often susceptible to proteolytic enzyme degradation, short circulating half-life, low solubility, and rapid clearance by the kidneys. Such peptides also have a propensity to generate neutralising antibodies. The process of PEGylation can circumvent problems associated with the use of peptide-based therapeutics. The resultant pharmacokinetic outcomes of PEGylation can manifest as changes occurring in overall circulation life span, tissue distribution pattern, and elimination pathway of the attached therapeutic molecule, which can ultimately result in improved pharmacodynamic outcomes.

PEGylation can prolong the circulatory half-life of a protein, allowing the protein to be effective over a longer time. The covalent attachment of PEG to a protein can significantly increase the protein's effective size and hydrodynamic volume, and so reduce its clearance rate from the body, especially via the kidneys. Similarly, the attached PEG molecule can act as a physical barrier to proteolytic enzymes, thereby reducing the enzymatic degradation of the PEGylated protein. However, PEGs are typically of a molecular weight of 20-40 kDa whilst, optionally, the polymer moiety used in the present invention has a molecular weight of no more than 1000 Da.

The invention provides a peptide based on biologically active CCK-8, the peptide having improved characteristics for the treatment of at least one of obesity and type 2 diabetes, wherein the structure of the peptide is:

(Z)-Asp¹-Aaa²(X)-Aaa³Gly⁴Trp⁵Aaa⁶Asp⁷(Y)Aaa⁸K,

wherein:
the amino acids may be either D or L amino acids;
the bond between amino acid residues is either a peptide bond or a non-peptide isostere bond;
Aaa² is selected from the group comprising Tyr and Phe;
when Aaa² is Tyr, X is selected from the group comprising SO₃H⁻, PO₃H₂⁻ and a polymer moiety of the general formula —O—(CH₂—O—CH₂)ₙ—H, in which n is an integer between 1 and about 22, wherein the X is covalently bound to the para phenyl oxygen of Tyr, and, when Aaa² is Phe, X is CH₂SO₃Na, wherein the X is covalently bound to the para phenyl position of Phe;
Aaa³ is selected from the group comprising Met, norleucine, 2-aminohexanoic acid and Thr;
Aaa⁶ is selected from the group comprising Met, norleucine, 2-aminohexanoic acid and Phe;
Aaa⁸ is selected from the group comprising Phe and Met;
(Y)Aaa⁸K, when Aaa⁸ is Phe⁸ and K is an amide, is:

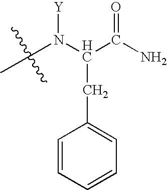

Y is covalently bound to nitrogen and is selected from the group consisting of H and CH₃;
K is selected from the group consisting of the hydroxyl group of Phe⁸, an amide covalently bound to Phe⁸, an ester covalently bound to Phe⁸, a salt of the hydroxyl group of Phe⁸, a salt of an amide covalently bound to Phe⁸, a salt of an ester covalently bound to Phe⁸ and a polymer moiety of the general formula —O—(CH₂—O—CH₂)ₙ—H, in which n is an integer between 1 and about 22;
Z comprises at least one amino acid modification, wherein said at least one modification comprises an N-terminal extension, or an N-terminal modification; and at least one of Z, X and K is a polymer moiety of the general formula —O—(CH₂—O—CH₂)ₙ—H, in which n is an integer between 1 and about 22.
Optionally, the peptide is further modified by attachment to Asp⁷ of a polymer moiety of the general formula HO—(CH₂—O—CH₂)ₙ—H, in which n is an integer between 1 and about 22. Further optionally, the peptide is modified by replacement of any amino acid with an amino acid selected from the group including, but not limited to, lysine, cysteine, histidine, arginine, aspartic acid, glutamic acid, serine, threonine, and tyrosine and attachment of a polymer moiety of the general formula HO—(CH₂—O—CH₂)ₙ—H, in which n is an integer between 1 and about 22 to at least one substituted amino acid. Optionally, at least one of K, X and Z comprises a polymer moiety covalently bound to Phe⁸, the polymer moiety being of the general formula —O—(CH₂—O—CH₂)ₙ—H, in which n is an integer between 1 and about 10; optionally, wherein n is an integer between about 2 and about 6.

Further optionally, at least one of X and K is a polymer moiety of the general formula —O—(CH₂—O—CH₂)ₙ—H, in which n is an integer between 1 and about 22 and Z is an N-terminal extension is selected from the group comprising pGlu, pGlu-Gln, an acid, a fatty acid, Boc, Fmoc and Arg. Still further optionally, at least one of X and K is a polymer moiety of the general formula —O—(CH₂—O—CH₂)ₙ—H, in which n is an integer between 1 and about 22 and Z is pGlu-Gln.

The invention further provides a peptide based on biologically active CCK-8, the peptide having improved characteristics for the treatment of at least one of obesity and type 2 diabetes, wherein the structure of the peptide is:

(Z)-Asp¹-Aaa²(X)-Aaa³Gly⁴Trp⁵Aaa⁶Asp¹(Y)Aaa⁸K,

wherein:
the amino acids may be either D or L amino acids;
the bond between amino acid residues is either a peptide bond or a non-peptide isostere bond;
Aaa² is selected from the group comprising Tyr and Phe;
when Aaa² is Tyr, X is selected from the group comprising SO₃H⁻, PO₃H₂ and a polymer moiety of the general formula —O—(CH₂—O—CH₂)ₙ—H, in which n is an integer between 1 and about 22, wherein the X is covalently bound to the para phenyl oxygen of Tyr, and, when Aaa² is Phe, X is CH₂SO₃Na, wherein the X is covalently bound to the para phenyl position of Phe;
Aaa³ is selected from the group comprising Met, norleucine, 2-aminohexanoic acid and Thr;
Aaa⁶ is selected from the group comprising Met, norleucine, 2-aminohexanoic acid and Phe;
Aaa⁸ is selected from the group comprising Phe and Met;
(Y)Aaa⁸K, when Aaa⁸ is Phe⁸ and K is an amide, is:

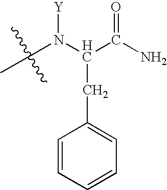

Y is covalently bound to nitrogen and is selected from the group consisting of H and CH₃;
K is selected from the group consisting of the hydroxyl group of Phe⁸, an amide covalently bound to Phe⁸, an ester covalently bound to Phe⁸, a salt of the hydroxyl group of Phe⁸, a salt of an amide covalently bound to Phe⁸, a salt of an ester covalently bound to Phe⁸ and a polymer moiety of the general formula —O—(CH₂—O—CH₂)ₙ—H, in which n is an integer between 1 and about 22; and
Z comprises at least one amino acid modification, said N-terminal modification at position 1 being selected from the group comprising N-alkylation, N-acetylation, N-acylation, N-glycation and N-isopropylation of the amino acid at position 1. Optionally, the N-terminal modification is N-acylation. Further optionally, the N-terminal modification is N-acetylation.

The invention further provides a peptide based on biologically active CCK-8, the peptide having improved characteristics for the treatment of at least one of obesity and type 2 diabetes, wherein the structure of the peptide is:

(Z)-Asp¹-Tyr²(PO₃H₂⁻)-Aaa³Gly⁴Trp⁵Aaa⁶Asp⁷(Y)Aaa⁸K,

wherein:
the amino acids may be either D or L amino acids;
the bond between amino acid residues is either a peptide bond or a non-peptide isostere bond;
Aaa³ is selected from the group comprising Met, norleucine, 2-aminohexanoic acid and Thr;
Aaa⁶ is selected from the group comprising Met, norleucine, 2-aminohexanoic acid and Phe;
Aaa⁸ is selected from the group comprising Phe and Met;
(Y)Aaa⁸K, when Aaa⁸ is Phe⁸ and K is an amide, is:

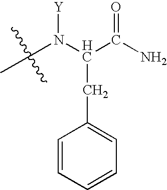

Y is covalently bound to nitrogen and is selected from the group consisting of H and CH₃;
K is selected from the group consisting of the hydroxyl group of Phe⁸, an amide covalently bound to Phe⁸, an ester covalently bound to Phe⁸, a salt of the hydroxyl group of Phe⁸, a salt of an amide covalently bound to Phe⁸, a salt of an ester covalently bound to Phe⁸ and a polymer moiety of the general formula —O—(CH₂—O—CH₂)ₙ—H, in which n is an integer between 1 and about 22; and
Z comprises at least one amino acid modification, said N-terminal modification at position 1 being selected from the group comprising N-alkylation, N-acetylation, N-acylation, N-glycation and N-isopropylation of the amino acid at position 1. Optionally, the N-terminal modification is N-acylation. Further optionally, the N-terminal modification is N-acetylation.

The invention further provides a fragment of the peptide of the invention, wherein the structure of the peptide fragment is:

(Z)-Aaa²(X)-Aaa³Gly⁴Trp⁵Aaa⁶Asp⁷(Y)Aaa⁸K,

wherein:
the amino acids may be either D or L amino acids;
the bond between amino acid residues is either a peptide bond or a non-peptide isostere bond;
Aaa² is selected from the group comprising Tyr and Phe;
when Aaa² is Tyr, X is selected from the group comprising SO₃H⁻, PO₃H₂⁻ and a polymer moiety of the general formula —O—(CH₂—O—CH₂)ₙ—H, in which n is an integer between 1 and about 22, wherein the X is covalently bound to the para phenyl oxygen of Tyr, and, when Aaa² is Phe, X is CH₂SO₃Na, wherein the X is covalently bound to the para phenyl position of Phe;
Aaa³ is selected from the group comprising Met, norleucine, 2-aminohexanoic acid and Thr;
Aaa⁶ is selected from the group comprising Met, norleucine, 2-aminohexanoic acid and Phe;
Aaa⁸ is selected from the group comprising Phe and Met;
(Y)Aaa⁸K, when Aaa⁸ is Phe and K is an amide, is:

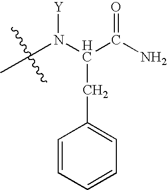

Y is covalently bound to nitrogen and is selected from the group consisting of H and CH₃;
K is selected from the group consisting of the hydroxyl group of Phe⁸, an amide covalently bound to Phe⁸, an ester covalently bound to Phe⁸, a salt of the hydroxyl group of Phe⁸, a salt of an amide covalently bound to Phe⁸, a salt of an ester covalently bound to Phe⁸ and a polymer moiety covalently bound to Phe⁸, the polymer moiety being of the general formula —O—(CH₂—O—CH₂)ₙ—H, in which n is an integer between 1 and about 22; and
Z comprises at least one amino acid modification, wherein said at least one modification comprises an N-terminal modification, said N-terminal extension being selected from the group comprising pGlu, pGlu-Gln, an acid, a fatty acid, Boc, Fmoc, Arg and attachment of a polymer moiety of the general formula —O—(CH₂—O—CH₂)ₙ—H, in which n is an integer between 1 and about 22. Optionally, the acid is

Further optionally, the structure of the peptide fragment is:

(Z)-Aaa²(X)-Aaa³Gly⁴Trp⁵ Aaa⁶Asp⁷(Y)Aaa⁸K,
wherein:
   the amino acids are L amino acids;
   the bonds between amino acid residues are peptide bonds;
   Aaa³ and Aaa⁶ are each Met;
   Aaa⁸ is Phe;
   Aaa²(X) is Tyr²(X):

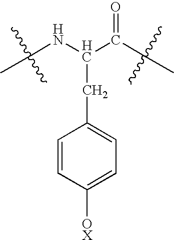

X is covalently bound to oxygen and selected from the group consisting of SO₃H⁻, PO₃H₂⁻ and a polymer moiety of the general formula —O—(CH₂—O—CH₂)ₙ—H, in which n is an integer between 1 and about 22;
K is an amide covalently bound to Phe⁸; and
Y is selected from the group consisting of H and CH₃.

Still further optionally, the invention provides a peptide fragment, wherein said N-terminal modification is selected from the group comprising N-alkylation, N-acetylation, N-acylation, N-glycation, or N-isopropylation at Aaa². Even still further optionally, Aaa² is Tyr and said N-terminal modification is selected from the group comprising:

(i) acetylation of Tyr²;
(ii) glycation of Tyr²; and
(iii) acylation of Tyr² by succinic acid.

Still further optionally, the invention provides a peptide fragment, wherein said N-terminal extension is selected from the group comprising pGlu, pGlu-Gln, an acid, a fatty acid, Boc, Fmoc, Arg and a polymer moiety of the general formula —O—(CH₂—O—CH₂)ₙ—H, in which n is an integer between 1 and about 22. Even still further optionally, wherein said N-terminal extension is selected from the group comprising:

(i) modification of Tyr² by pyroglutamyl;
(ii) modification of Tyr² by Fmoc; and
(iii) modification of Tyr² by Boc.

A further aspect of the invention provides use of at least one of the aforementioned peptides and peptide fragments in the preparation of a medicament to at least one of inhibit food intake, induce satiety, stimulate insulin secretion, moderate blood glucose excursions, enhance glucose disposal and exhibit enhanced stability in plasma compared to native CCK-8.

A further aspect of the invention provides use at least one of the aforementioned peptides and peptide fragments or Asp¹-glucitol CCK-8 in the preparation of a medicament for the treatment of at least one of obesity and type 2 diabetes.

A further aspect of the invention provides a pharmaceutical composition including at least one of the aforementioned peptides and peptide fragments.

A further aspect of the invention provides a pharmaceutical composition useful in the treatment of at least one of obesity and type 2 diabetes, which comprises an effective amount of at least one of the aforementioned peptides and peptide fragments in admixture with a pharmaceutically acceptable excipient for delivery through transdermal, nasal inhalation, oral or injected routes. Optionally, the pharmaceutical composition further comprises native or derived analogues of leptin, exendin, islet amyloid polypeptide (IAPP) or bombesin.

A further aspect of the invention provides a method for treating at least one of obesity and type 2 diabetes, the method comprising administering to an individual in need of such treatment an effective amount at least one of the aforementioned peptides and peptide fragments.

The invention will now be demonstrated with reference to the following non-limiting examples and the accompanying figures wherein:

FIG. 1 illustrates the degradation of CCK-8 and Asp¹-glucitol CCK-8 by plasma.

FIG. 2 illustrates the lack of degradation of pGlu-Gln CCK-8 by plasma.

FIG. 3 illustrates the effect of CCK-8, Asp¹-glucitol CCK-8 and pGlu-Gln CCK-8 on food intake.

FIG. 4 illustrates the effect of CCK-8 and Asp¹-glucitol CCK-8 on food intake in ob/ob mice.

FIG. 5 illustrates the effect of different doses of CCK-8 on food intake.

FIG. 6 illustrates the effect of different doses of Asp¹-glucitol CCK-8 on food intake.

FIG. 7 illustrates the effect of different doses of pGlu-Gln CCK-8 on food intake.

FIG. 8 illustrates the effect of CCK-8 and leptin both alone and combined on food intake.

FIG. 9 illustrates the effect of CCK-8 and IAPP both alone and combined on food intake.

FIG. 10 illustrates the effect of bombesin and pGlu-Gln CCK-8 on food intake.

FIG. 11 illustrates the effect of pGlu-Gln CCK-8 and leptin both alone and combined on food intake.

FIG. 12 illustrates the effect of pGlu-Gln CCK-8 and leptin both alone and combined on food intake.

FIG. 13 illustrates the extensive degradation of CCK-8 to N-terminally truncated forms when incubated with mouse plasma for 120 min.

FIG. 14 illustrates lack of degradation of N—Ac—CCK-8 when incubated with mouse plasma for 120 min.

FIG. 15 illustrates the protracted dose-dependent inhibitory effects of N—Ac—CCK-8 on feeding in normal mice.

FIG. 16 illustrates the inhibitory effects of pGluGln-CCK-8 on feeding activity in ob/ob mice on days 1 and 7 of daily dosing.

FIG. 17 illustrates body weight reduction in high fat fed obese mice treated daily with pGluGln-CCK-8.

FIG. 18 illustrates decrease of non-fasting glucose concentrations at 09.00-21.00 h in high fat fed obese mice treated daily with pGluGln-CCK-8.

FIG. 19 illustrates lower glycaemic excursion following feeding in high fat fed obese mice treated daily with pGluGln-CCK-8.

FIG. 20 illustrates improved glucose tolerance in high fat fed obese mice treated daily with pGluGln-CCK-8.

FIG. 21 illustrates enhanced insulin sensitivity in high fat fed obese mice treated daily with pGluGln-CCK-8.

FIG. 22 illustrates body weight reduction in high fat fed obese mice treated daily with pGluGln-CCK-8 or pGluGln-CCK-8-Peg.

FIG. 23 illustrates inhibition of food intake in high fat fed obese mice treated daily with pGluGln-CCK-8 or pGluGln-CCK-8-Peg.

FIG. 24 illustrates improvement of intraperitoneal glucose tolerance in high fat fed obese mice treated daily with pGluGln-CCK-8 or pGluGln-CCK-8-Peg.

FIG. 25 illustrates the improvement of oral glucose tolerance in high fat fed obese mice treated daily with pGluGln-CCK-8 or pGluGln-CCK-8-Peg.

FIG. 26 illustrates improved insulin sensitivity in high fat fed obese mice treated daily with pGluGln-CCK-8 or pGluGln-CCK-8-Peg.

FIG. 27 illustrates long-lasting effects of pGluGln-CCK-8 and especially pGluGln-CCK-8-Peg on inhibition of feeding when administered acutely to high fat fed obese mice.

FIG. 28 illustrates that long-lasting effects of pGluGln-CCK-8 and especially pGLuGln-CCK-8-Peg on inhibition of feeding when administered 18 h previously to high fat fed obese mice.

FIG. 29 illustrates ineffectiveness of phosphorylated and non-sulphated, as opposed to the native sulphated, form of CCK-8 as inhibitor of feeding in mice.

FIG. 30 illustrates powerful stimulatory effects of phosphorylated CCK-8 and pGluGln-CCK-8 on insulin secretion from the clonal pancreatic beta cell line, BRIN-BD11.

### EXAMPLE 1

### Preparation of N-Terminally Modified CCK-8 and Analogues Thereof

The N-terminal modification of CCK-8 is essentially a three-step process. Firstly, CCK-8 is synthesised from its C-terminal (starting from an Fmoc-Phe-OCH₂-PAM-Resin, Novabiochem) up to Met³ on an automated peptide synthesizer (Applied Biosystems, CA, USA). The synthesis follows standard Fmoc peptide chemistry protocols utilizing other protected amino acids in a sequential manner used including Fmoc-Asp(OtBu)-OH, Fmoc-Met-OH, Fmoc-Trp-OH, Fmoc-Gly-OH, Fmoc-Met-OH. Deprotection of the N-terminal Fmoc-Met will be performed using piperidine in DMF (20% v/v). The OtBu group will be removed by shaking in TFA/Anisole/DCM. Secondly, the penultimate N-terminal amino acid of native CCK-8 (Tyr(tBu) is added to a manual bubbler system as an alkali labile Fmoc-protected Tyr(tBu)-PAM resin. This amino acid is deprotected at its N-terminus (piperidine in DMF (20% v/v)). This is then allowed to react with excess Fmoc-Asp(OtBu)-OH forming a resin bound dipeptide Fmoc-Asp(OtBu)-Tyr(tBu)-PAM resin. This will be deprotected at its N-terminus (piperidine in DMF (20% v/v)) leaving a free α-amino group. This will be allowed to react with excess glucose (glycation, under reducing conditions with sodium cyanoborohydride), acetic anhydride (acetylation), pyroglutamic acid (pyroglutamyl) etc. for up to 24 hours as necessary to allow the reaction to go to completion. The completeness of reaction will be monitored using the ninhydrin test which will determine the presence of available free α-amino groups. Deprotection of the side-chains will be achieved by shaking in TFA/Anisole/DCM. Sulphation of the N-terminally modified dipeptide will be achieved by suspending the peptide in DMF/pyridine and adding sulphur trioxide-pyridine complex with shaking up to 24 hours. Once the reaction is complete, the now structurally modified N-terminal dipeptide, containing the sulphated Tyr, will be cleaved from the PAM resin (under basic conditions with methanolic ammonia) and with appropriate scavengers. Thirdly, a 4-fold excess of the N-terminally modified-Asp-Tyr(SO₃H)—OH will be added directly to the automated peptide synthesizer, which will carry on the synthesis, thereby stitching the N-terminally modified-region to the α-amino of CCK(Met³), completing the synthesis of the sulphated CCK analogue. This peptide is cleaved off the PAM resin (as above under alkaline conditions) and then worked up using the standard Buchner filtering, precipitation, rotary evaporation and drying techniques. The filtrate will be lyophilized prior to purification on a Vydac semi-preparative C-18 HPLC column (1.0×25 cm). Confirmation of the structure of CCK-8 related analogues will be performed by mass spectrometry (ESI-MS and/or MALDI-MS).

### EXAMPLE 2

### Effects of CCK-8 Analogues on Food Intake

The following example investigates preparation of Asp¹-glucitol CCK-8 and pGlu-Gln CCK-8 together with evaluation of their effectiveness at inducing satiety and decreasing food intake in vivo. The results clearly demonstrate that these novel analogues exhibit substantial resistance to aminopeptidase degradation and increased biological activity compared with native CCK-8.

### Research Design and Methods

### Materials.

Cholecystokinin octapeptide (sulphated CCK-8), pGlu-Gln CCK-8 and other analogues will be synthesised using an Applied Biosystems 432 Peptide synthesizer (as described above). HPLC grade acetonitrile was obtained from Rathburn (Walkersburn, Scotland). Sequencing grade trifluoroacetic acid (TFA) was obtained from Aldrich (Poole, U.K.). All water used in these experiments was purified using a Milli-Q, Water Purification System (Millipore Corporation, Millford, Mass., U.S.A.). All other chemicals purchased were from Sigma, Poole, UK.

Preparation of Asp¹Glucitol CCK-8 and pGlu-Gln CCK-8.

Asp¹-glucitol CCK-8 and pGlu-Gln CCK-8 were prepared by a 3 step process as described in Example 1. The peptides were purified on a Vydac semi-preparative C-18 HPLC column (1.0×25 cm) followed by a C-18 analytical column using gradient elution with acetonitrile/water/TFA solvents. Confirmation of the structure of CCK-8 related analogues was by mass spectrometry (ESI-MS and/or MALDI-MS). Purified control and structurally modified CCK-8 fractions used for animal studies were quantified (using the Supelcosil C-8 column) by comparison of peak areas with a standard curve constructed from known concentrations of CCK-8 (0.78-25 μg/ml).

Molecular Mass Determination of Asp¹Glucitol CCK-8 and pGlu-Gln CCK-8 by Electrospray Ionization Mass Spectrometry (ESI-MS).

Samples of CCK-8 and structurally modified CCK-8 analogues were purified on reversed-phase HPLC. Peptides were dissolved (approximately 400 pmol) in 100 μl of water and applied to the LCQ benchtop mass spectrometer (Finnigan MAT, Hemel Hempstead, UK) equipped with a microbore C-18 HPLC column (150×2.0 mm, Phenomenex, UK, Ltd., Macclesfield). Samples (30 μl direct loop injection) were injected at a flow rate of 0.2 ml/min, under isocratic conditions 35% (v/v) acetonitrile/water. Mass spectra were obtained from the quadripole ion trap mass analyzer and recorded. Spectra were collected in the positive and negative mode using full ion scan mode over the mass-to-charge (m/z) range 150-2000. The molecular masses of positive ions from CCK-8 and related analogues were determined from ESI-MS profiles using prominent multiple charged ions and the following equation Mᵣ=iMᵢ−iMₕ (where Mᵣ=molecular mass; Mᵢ=m/z ratio; i=number of charges; Mₕ=mass of a proton).

Effects of CCK-8, Asp¹Glucitol CCK-8, pGlu-Gln CCK-8 and Other Peptides on Food Intake in Mice.

Studies to evaluate the relative potencies of control CCK-8, Asp¹-glucitol CCK-8, pGlu-Gln CCK-8 and other peptides involved in regulation of feeding were performed using male Swiss TO mice (n=16) aged 7-12 weeks from a colony originating from the Behavioral and Biomedical Research Unit, University of Ulster. The animals were housed individually in an air-conditioned room at 22+−2° C. with 12 h light/12 h dark cycle. Drinking water was supplied ad libitum and standard mouse maintenance diet (Trouw Nutrition, Cheshire, UK) was provided for various times as indicated below. The mice were habituated to a daily feeding period of 3 h/day by progressively reducing the feeding period over a 3 week period. On days 1-6, food was supplied from 10.00 to 20.00 h, days 7-14 from 10.00 to 16.00 h and days 15-21 food was restricted to 10.00 to 13.00 h. Body weight, food and water intake were monitored daily.

Mice which had been previously habituated to feeding for 3 h/day were administered a single i.p. injection of saline (0.9% w/v NaCl, 10 ml/kg) in the fasted state (10.00 h) and food was immediately returned following injection. Two days after the saline injection, mice were randomly allocated into groups of 7-8 animals which were administered a single i.p. injection (from 1 to 100 nmol/kg) of either CCK-8, structurally modified CCK-8 analogues and/or other peptide hormones (including, bombesin, leptin and islet amyloid polypeptide (IAPP)). Food intake was carefully monitored at 30 min intervals up to 180 min post injection. In one series of experiments, the ability of CCK-8 and Asp¹-glucitol CCK-8 to inhibit feeding activity was studied in overnight fasted adult obese hyperglycaemic (ob/ob) mice. All animal studies were done in accordance with the Animals (Scientific Procedures) Act 1986.

Effects of Mouse Serum on Degradation of CCK-8, Asp¹Glucitol CCK-8 and pGlu-Gln CCK-8.

Serum (20 μl) from fasted Swiss TO mice was incubated at 37° C. with 10 μg of either native CCK-8, Asp¹-glucitol CCK-8 or pGlu-Gln CCK-8 for periods up to 2 h in a reaction mixture (final vol. 500 μl) containing 50 mmol/l triethanolamine/HCl buffer pH 7.8. The reaction was stopped by addition of 5 μl of TFA and the final volume adjusted to 1.0 ml using 0.1% (v/v) TFA/water. Samples were centrifuged (13,000 g, 5 min) and the supernatant applied to a C-18 Sep-Pak cartridge (Waters/Millipore) which was previously primed and washed with 0.1% (v/v) TFA/water. After washing with 20 ml 0.12% TFA/water, bound material was released by elution with 2 ml of 80% (v/v) acetonitrile/water and concentrated using a Speed-Vac concentrator (AES 1000, Savant). The volume was adjusted to 1.0 ml with 0.12% (v/v) TFA/water and applied to a (250×4.6 mm) Vydac C-18 column pre-equilibrated with 0.12% (v/v) TFA/water at a flow rate of 1.0 ml/min. The concentration of acetonitrile in the eluting solvent was raised from 0 to 31.5% over 15 min, from 31.5 to 38.5% over 30 min, and from 38.5 to 70% over 5 min, using linear gradients monitoring eluting peaks at 206 nm.

### Statistical Analysis.

Groups of data are presented as means+−SE. Statistical evaluation was performed using analysis of variance, least significant difference multiple comparisons test and Student's unpaired t-test as appropriate. Differences were considered to be significant if P<0.05.

### Results

### Molecular Mass Determination.

Following incubation, Asp¹-glucitol CCK-8 and pGlu-Gln CCK-8 were clearly separated from native CCK-8 on a Vydac C-18 HPLC column. The average molecular masses of CCK-8 (Mᵣ 1064.2), Asp¹-glucitol CCK-8 (Mᵣ 1228.4) and pGlu-Gln CCK-8 (Mᵣ 1352.4) were determined by ESI-MS, confirming their structures.

In Vitro Degradation of CCK-8, Asp¹Glucitol CCK-8 and pGlu-Gln CCK-8.

FIG. 1 shows a comparison of typical examples of HPLC traces following the action of mouse serum in vitro on the degradation of CCK-8 (left panels) or Asp¹glucitol CCK-8 (right panels) at time 0, 1 and 2 h. Intact CCK-8 (peak A) and three separate fragments of CCK-8 (peaks B, C, D) eluted at 22.18, 22.01, 19.81 and 18.98 min, respectively. Asp¹glucitol CCK-8 (peak E, right panels) eluted at 21.65 min. Table 1 summarises the pattern of CCK-8 and Asp¹glucitol CCK-8 breakdown in each case. From analysis of HPLC peak area data it is evident that 83.1% and 100% of the CCK-8 was converted to the CCK-8 fragments after 1 and 2 h incubation, respectively. In contrast, Asp¹-glucitol CCK-8 remained intact after 1 and 2 h incubation and no additional peptide fragments were detected. Similarly, pGlu-Gln CCK-8 was also highly resistant to plasma degradation after 2 h (FIG. 2).

### Food Intake Trials.

The daily food intake of mice during the period before administration of peptides indicated that mean food consumption of the mice allowed 3 h access to food was 3.8+−0.2 g/mouse. Following administration of i.p. saline, there was no significant difference in 3 h voluntary food intake (3.66+−0.1 g) when compared to 3 h food intake alone. FIG. 3 shows that i.p. injection with CCK-8 had an inhibitory effect on voluntary food intake at 30, 60 and 90 min post treatment compared to saline alone. However, there was no sustained inhibitory action of CCK-8 on cumulative food intake beyond 90 min. In contrast, the inhibitory effect of Asp¹-glucitol CCK-8 and pGlu-Gln CCK-8 on food intake was sustained over the 3 h post-treatment feeding period compared to saline response. Furthermore, both structurally modified CCK-8 peptides were significantly more potent at reducing food intake at each time point (except at 30 min) compared to the equivalent dose of CCK-8. FIG. 4 shows that CCK-8 and Asp¹-glucitol CCK-8 also significantly reduce voluntary food intake in genetically obese diabetic (ob/ob) mice. Asp¹-glucitol CCK-8 is considerable more potent than native CCK-8.

Dose-response effects of CCK-8, Asp¹-glucitol CCK-8 and pGlu-Gln CCK-8 on food intake are shown in FIGS. 5-7. Compared with CCK-8, both structurally modified peptides exerted more prolonged effects at lower doses. As shown in FIGS. 8-10, CCK-8 or pGlu-Gln CCK-8 were considerably more potent on equimolar basis than either leptin, islet amyloid polypeptide (IAPP) or bombesin in inhibiting food intake over a 30-180 min period. Combination of CCK-8 with either leptin or IAPP, particularly the latter, resulted in a very marked potentiation of satiety action (FIGS. 8-9). FIG. 10 shows that both pGlu-Gln CCK-8 and bombesin are effective anorectic agents but that the former has longer lasting effects. FIG. 11 shows that combination of CCK-8 with exendin(1-39) has particularly enhanced satiety action. Administration of leptin with pGlu-Gln CCK-8 also resulted in a particularly marked and long-lasting inhibition of food intake.

### Discussion

The current study examined the effects of CCK-8, Asp¹-glucitol CCK-8 and pGlu-Gln CCK-8 on food intake in mice. The present study demonstrated that CCK-8 was effective in reducing food intake up to 90 min after administration compared to saline controls. The effects of Asp¹-glucitol CCK-8 and pGlu-Gln CCK-8 on food intake were investigated and revealed that these amino-terminally modified peptides had a remarkably enhanced and prolonged ability to reduce voluntary food intake compared to an equimolar dose of native CCK-8. The alteration in primary structure by N-terminal modification of CCK-8 appears to enhance its biological activity and extend its duration of action in normal animals from 90 min to more than 3 h. Indeed the results also indicate that a potent satiety effect can persist for more than 5 h in obese diabetic (ob/ob) mice. The change in biological activity encountered with Asp¹-glucitol CCK-8 and pGlu-Gln CCK-8 extends previous observations that glycation of peptides can alter their biological activities. It is noteworthy that control experiments conducted with glycated tGLP-1 indicate that the presence of a glucitol adduct on the amino-terminus of a peptide, is insufficient on its own to induce satiety in this test system.

The fact that Asp¹-glucitol CCK-8 and pGlu-Gln CCK-8 enhance appetite suppression raises the question of a possible mechanism. Since the very short 1-2 min half-life of CCK-8 is generally accepted as the explanation of the transient satiety effect of the peptide, it is possible that modification of the amino terminus of CCK-8 prolongs the half-life by protecting it against aminopeptidase attack thus enhancing its activity. Aminopeptidase A has been shown to directly degrade CCK-8 in vivo by hydrolysing the Asp-Tyr bond. The peptide can also be degraded by neutral endopeptidase 24.11 (NEP), thiol or serine endopeptidases and angiotensin converting enzyme. The present study revealed that Asp¹-glucitol CCK-8 and pGlu-Gln CCK-8 were extremely resistant to degradation by peptidases in serum. Thus it seems likely that protection of the amino terminus of CCK-8 with a glucitol or pyroglutamyl-Gln adduct enhances the half-life of glycated CCK-8 in the circulation and thus contributes to enhancement of its biological activity by extending its duration of action in vivo.

Various mechanisms have been proposed to explain the action of CCK in reducing food intake. One hypothesis is that after ingestion of food, gastric distension and nutrient absorption causes release of CCK-8 which ends feeding. It is proposed that CCK-8 both contracts the pyloric sphincter as well as relaxing the proximal stomach which together delays gastric emptying. The gastric branch of the vagus nerve is closely involved in mediating the action of CCK-8. The satiety signal appears to be transmitted from the vagus nerve to the hypothalamus via the nucleus tractus solitarius and the area postrema.

Although much attention has been given to actions and possible therapeutic use of leptin in obesity and NIDDM, Asp¹-glucitol CCK-8, pGlu-Gln CCK-8 or other structurally modified analogues of CCK-8 may potentially have a number of significant attributes compared with leptin. Firstly, there is accumulating evidence for defects in the leptin receptor and post-receptor signalling in certain forms of obesity-diabetes. Secondly, CCK-8 has potent peripheral actions, whereas leptin acts centrally and requires passage through the blood-brain barrier. Thirdly, the effects of CCK-8 on food intake are immediate whereas the action of leptin requires high dosage and is protracted. Fourthly, CCK has been shown to act as a satiety hormone in humans at physiological concentrations and a specific inhibitor of CCK degradation demonstrates pro-satiating effects in rats. It is also interesting to note that the effects of CCK-8 administered together with either leptin, IAPP, exendin(1-39) or bombesin on satiety are additive, raising the possibility of complementary mechanisms and combined therapies.

In summary, this study demonstrates that CCK-8 can be readily structurally modified at the amino terminus and that intraperitoneally administered Asp¹-glucitol CCK-8 or pGlu-Gln CCK-8, in particular, display markedly enhanced satiating action in vivo, due in part to protection from serum aminopeptidases. These data clearly indicate the potential of N-terminally modified CCK-8 analogues for inhibition of feeding and suggest a possible therapeutic use in humans in the management of obesity and related metabolic disorders.

TABLE 1
Effect of serum on in vitro degradation of CCK-8 and glycated CCK-8

| Incubation | | Peak Retention | % Total CCK- |
|---|---|---|---|
| Time(h) | Peak Identity | Time (min) | like material |
| CCK-8 | | | |
| 0 | CCK-8 (A) | 22.18 | 100 |
| 1 | CCK-8 fragment (C) | 19.81 | 43.8 |
| | CCK-8 fragment (B) | 22.01 | 39.3 |
| | CCK-8 (A) | 22.18 | 16.9 |
| 2 | CCK-8 fragment (D) | 18.98 | 11.8 |
| | CCK-8 fragment (C) | 19.81 | 29.5 |
| | CCK-8 fragment (B) | 22.01 | 58.7 |
| | CCK-8 (A) | 22.18 | 0 |
| Glycated CCK-8 | | | |
| 0 | Glycated CCK-8 | 21.65 | 100 |
| 1 | Glycated CCK-8 | 21.65 | 100 |
| 2 | Glycated CCK-8 | 21.65 | 100 |

We have described that N-terminal modification of CCK-8 endows the molecule with resistance to in vivo enzymatic degradation, thereby substantially increasing its potency as a satiety agent and potential therapeutic agent. Claims were made for a range of N-terminal modifications together with beneficial combinations with other obesity or diabetic drugs. Here we present supporting data to exemplify and extend those claims. This work fully supports the utility of analogues of CCK-8 for treatment of obesity and diabetes. These data show stability/effectiveness of another N-terminal modification —N—Ac—CCK-8; illustrate effects going beyond normal mice, i.e., animals with genetic or diet-induced obesity; demonstrate that inhibition of food intake is sustainable and able to induce significant body weight loss; demonstrate absence of toxic or adverse effects on welfare of animals dosed twice per day for up to 34 days; evidence benefit of 2nd generation modification, i.e., using long-acting-PEGylation; show beneficial effects not only on food intake, body weight but on various parameters of blood glucose control; demonstrate that phosphorylated CCK-8 is an unexpected stimulator of insulin secretion—possibly with therapeutic potential; and show that CCK-8 and pGluGln-CCK-8 stimulate insulin secretion.

The invention will now be demonstrated with reference to the following non-limiting examples and the accompanying figures wherein:

### Methods

Peptide synthesis: CCK-8 peptides (sulphated form, unless indicated otherwise) were sequentially synthesised with an automated peptide synthesiser using standard solid phase Fmoc procedure. Peptides were purified by reversed-phase HPLC using Vydac analytical columns (The Separations Group, Hesperia, USA). The structure of purified peptides was confirmed by mass spectrometry.

Degradation of CCK-8 and related peptides: To assess the susceptibility of CCK-8 peptides to in vivo degradation, serum (20 μl) from fasted Swiss TO mice was incubated at 37° C. with 10 μg of peptide for various times in a reaction mixture (final vol. 500 μl) containing 50 mmol/l triethanolamine/HCl buffer pH 7.8. The reaction was stopped by addition of 5 μl of TFA and the final volume adjusted to 1.0 ml using 0.1% v/v TFA/water. Samples were centrifuged (13,000 g, 5 min) and the supernatant applied to a C-18 Sep-Pak cartridge (Waters/Millipore) which was previously primed and washed with 0.1% v/v TFA/water. After washing with 20 ml 0.12% TFA/water, bound material was released by elution with 2 ml of 80% v/v acetonitrile/water and concentrated using a Speed-Vac concentrator (AES 1000, Savant). The volume was adjusted to 1.0 ml with 0.12% TFA/water and applied to a (250×4.6 mm) Vydac C-18 column pre-equilibrated with 0.12% TFA/water at a flow rate of 1.0 ml/min. The concentration of acetonitrile in the eluting solvent was raised from 0 to 31.5% over 15 min, from 31.5 to 38.5% over 30 min, and from 38.5 to 70% over 5 min, using linear gradients monitoring eluting peaks at 206 nm. The identity of purified peptides was confirmed by mass spectrometry.

Molecular mass determination of CCK-8 peptides: MALDI-TOF mass spectrometry was carried out using out using a Voyager DE-PRO instrument (Applied Biosystems, Foster City, Calif., USA) that was operated in reflectron mode with delayed extraction. The accelerating voltage in the ion source was 20 kV and α-cyano-4-hydroxycinnamic acid was used as matrix. The instrument was calibrated with peptides of known molecular mass in the 2000-4000 Daltons range. The accuracy of mass determinations was ±0.02%.

pGluGlnCCK-8-PEG (PEG is the covalent attachment of a polymer moiety of the general formula HO—(CH₂—O—CH₂)ₙ—H, in which n is an integer between 1 and about 22)
Structure: pGlu-Gln-Asp-Tyr(SO₃H)-Met-Gly-Trp-Met-Asp-Phe-PEG

### Molecular Weight: 1630.7 amu

### Peptide Purity: 99.0%

N—Ac—CCK-8 (Ac is acetyl)

### Structure: Ac-Asp-Tyr(SO₃H)-Met-Gly-Trp-Met-Asp-Phe-NH₂

### Molecular Weight: 1185.3 amu

### Peptide Purity: 97.0%

CCK-8, where X is PO₃H₂

### Structure: Asp-Tyr(PO₃H₂)-Met-Gly-Trp-Met-Asp-Phe-NH₂

### Molecular Weight: 1143.3 Da

Culture of insulin-secreting cells: Clonal rat insulin-secreting BRIN-BD11 cells were cultured in RPMI-1640 tissue culture medium containing 10% (v/v) foetal calf serum, 1% (v/v) antibiotics (100 U/ml penicillin, 0.1 mg/ml streptomycin) and 11.1 mM glucose. The production and characterisation of BRIN-BD11 cells are described elsewhere (McClenaghan et al., 1996). Cells were maintained in sterile tissue culture flasks (Corning, Glass Works, UK) at 37° C. in an atmosphere of 5% CO2 and 95% air using LEEC incubator (Laboratory Technical Engineering, Nottingham, UK). Cell monolayers were used to assess insulin release. The cells were harvested with the aid of trypsin/EDTA (Gibco), seeded into 24-multiwell plates (Nunc, Rosklide, Denmark) at a density of 1.5×106 cells per well, and allowed to attach overnight. Prior to acute test, cells were preincubated for 40 min at 37° C. in a 1.0 ml Krebs Ringer bicarbonate buffer (115 mM NaCl, 4.7 mM KCl, 1.28 mM CaCl2, 1.2 mM KH2PO4, 1.2 mM MgSO4, 10 mM NaHCO3, 5 g/l bovine serum albumin, pH 7.4) supplemented with 1.1 mM glucose. Test incubations were performed for 20 min at 37° C. using the same buffer supplemented with 5.6 mM glucose in the absence (control) and presence of various peptide concentrations. The phosphodiesterase inhibitor, IBMX, was added to preserve cyclic AMP and enhance the natural secretory effects of CCK-8. Insulin was measured by radioimmunoassay.

Animal studies: Initial studies to evaluate the effects of CCK-8 peptides on feeding activity were performed using male Swiss TO mice (aged 7-12 weeks). Other studies used adult ob/ob mice (aged 12-16 weeks). The animals were housed individually in an air-conditioned room at 22±2° C. with 12 h light/dark cycle (08.00-20.00 h light). Drinking water was supplied ad libitum and standard mouse maintenance diet (Trouw Nutrition, Cheshire, UK) was provided as indicated. This normal mouse maintenance diet contains 3.5% fat, 14% protein and 63.9% carbohydrate; 4.5% fibre, crude oil 4.00%, ash 4.7%, and various minerals, amino acids and vitamins makes up the remainder and has a total metabolisable energy content is 13.1 kj/g. In other studies, TO mice were fed synthetic energy-rich high fat diet (45% fat, 20% protein and 35% carbohydrate; percent of total energy of 26.15 kj/g; Special Diets Service, Essex, UK) for up to 35 weeks to induce obesity and glucose intolerance. Some feeding experiments were performed using animals maintained on reverse light cycle (08.00-20.00 h dark).

Acute animal studies: Where indicated, TO mice were gradually habituated to a strict daily feeding regime of 3 h/day by progressively reducing the feeding time over a 3-week period. On days 1-6, food was supplied from 10:00 h to 20:00 h; on days 7-14, food was supplied from 10:00 h to 16:00 h; and on days 15-21 food was supplied from 10:00 h to 13:00 h. This was followed by one week of consistent 3 h daily food intake in which mice received a single i.p injection of saline (0.9% w/v NaCl; 10 ml/kg). For food intake studies, mice habituated to the feeding regime of 3 h/day were randomly allocated into groups. All peptides were dissolved in saline and administered intraperitoneally at the doses described in the legends. Food intake was monitored at 30 min intervals following introduction of food.

Long-term animal studies: Mice allowed unrestricted access to food were injected intraperitoneally with either peptide or saline (control) as described in the Figures. Food intake, body weight and indicators of blood glucose control (glucose tolerance, insulin sensitivity etc) were measured as indicated in the Figures and legends. All animal studies were carried out in accordance with the Animals (Scientific Procedures) Act of 1986.

Determination of plasma glucose and insulin: Plasma glucose concentration was measured by means of an automated glucose oxidase method using a Beckman Glucose Analyzer (Beckman Instruments, UK). Insulin was determined by radioimmunoassay.

Statistical analysis: Results are expressed as mean±S.E.M. Data were compared using Student's t-test or ANOVA followed by a Student-Newman-Keuls post hoc test, as appropriate. Groups of data were considered to be significantly different if P<0.05.

### Results and Discussion:

Consistent with our previous observations, HPLC combined with MALDI-TOF mass spectrometry revealed the rapid and extensive degradation of naturally occurring sulphated CCK-8 by incubation with mouse plasma for 120 min (FIG. 13). Fragment peptides were separated by reversed-phase HPLC and molecular masses identified by quadripole time of flight (Q-TOF) mass spectrometry —CCK-8, CCK-7, CCK-6 and CCK-5 are indicated by the arrows. In contrast, N—Ac—CCK-8 was entirely stable to degradation by plasma proteases, remaining totally intact at 120 min incubation (FIG. 14). This serves to illustrate our original claim that N-terminal modifications additional to those producing N-glucitol-CCK-8 and pGluGln-CCK-8, confer substantial biological stability and extended circulating half-life on CCK-8. Consistent with this view, N—Ac—CCK-8 displayed great and long-lasting potency in inhibiting voluntary food intake in normal mice habituated previously to 3 h feeding regimen (FIG. 15).

With this further indication to the efficacy of N-terminally modified CCK-8 analogues, a series of experiments was initiated to examine the effectiveness of these analogues in an animal model of genetic obesity-diabetes rather than in normal mice. This showed that daily administration of pGluGln-CCK-8 significantly inhibited food intake for more than 5 hours after injection (FIG. 16). Furthermore, the potency of this effect was similar on the first and seventh day of injecting, indicating that such a regimen was not associated with desensitisation of the CCK receptor.

Having shown efficacy of stable CCK-8 analogues in genetic obesity-diabetes, experiments were performed using normal mice previously maintained on a synthetic high fat energy-rich diet to induce obesity, insulin resistance and glucose intolerance. Such a model more closely resembles the obesity syndromes commonly observed in man. As expected, treatment of such animals with twice daily injection of pGluGln-CCK-8 resulted in substantial body weight loss due to decreased food intake over a period of more than 30 days (FIG. 17). This was associated with notable improvements of blood glucose control, including significant decrease of non-fasting glucose (FIG. 18), lower glycaemic excursion following feeding (FIG. 19), improved glucose tolerance (FIG. 20) and enhanced insulin sensitivity (FIG. 21). These observations point to an important antidiabetic action of stable CCK-8 analogues in addition to their utility to induce satiety and promote body weight loss.

The basic observations made using pGluGln-CCK-8 were fully confirmed by a separate series of experiments in high fat fed mice, which were designed to evaluate the effectiveness of a second generation analogue modified further by PEGylation to augment in vivo potency and in particular durability of biological activity. Twice daily administration of pGluGln-CCK-8-PEG reproduced all of the beneficial effects of pGluGln-CCK-8 on feeding activity, body weight, blood glucose control and insulin sensitivity (FIGS. 22-26). Comparison of the effectiveness of pGluGln-CCK-8-PEG, with the parent pGluGln-CCK-8 molecule, did not reveal much difference when given in twice daily injections. However, studies designed specifically to test durability of action against native CCK-8 in terms of inhibition of feeding showed that pGluGln-CCK-8-PEG was much more effective than pGluGln-CCK-8 (FIGS. 27-28). Notably, both peptides were able to inhibit feeding 21 hours after a single injection, clearing demonstrating the potential of such analogues of CCK-8 for treatment of obesity and related diabetes in man.

All of the peptides tested in these experiments were based on the naturally occurring sulphated form of CCK-8. Thus removal of the sulphate group resulted in substantial loss of biological activity in terms of inhibition of feeding as shown in FIG. 29. As a further innovation, we looked to see if substitution of the phosphate group would restore activity of CCK-8. This form of CCK-8 is much more readily synthesised than the sulphated form and additionally we noticed that it was much more stable to in vitro manipulations. However, this form completely lacked effects on feeding activity in parallel experiments (FIG. 29). In sharp contrast, and totally unexpectedly, in vitro studies using clonal BRIN-BD11 pancreatic beta cells revealed that phosphorylated CCK-8 was as potent as native (sulphated) CCK-8 or pGluGln-CCK-8 in stimulating insulin secretion (FIG. 30). One possible explanation is that the phosphorylated CCK-8 could possibly be acting on 2 different receptors here (in cell line on the beta-cells of pancreas and a different one in live animals on the vagus nerve). CCK1 and CCK2 receptors exist but their exact distribution in the body is not completely known.

These observations not only evidence the ability of these modified CCK-8 peptides to serve as potent stimulators of insulin secretion, but illustrate that phosphorylated CCK-8 and analogues thereof represent a class of potential new CCK drugs with differential effects on feeding and insulin secretory activity. The insulin output induced by these peptides is approximately equivalent to that induced by the therapeutic incretin hormones glucagon-like peptide-1 (GLP-1) and gastric inhibitory polypeptide (GIP).

Overall, this research further exemplifies the potential of stable N-terminally modified analogues of CCK-8 for promotion of satiety, body weight loss and improvement of blood glucose control. Molecules such as N—Ac—CCK-8 and pGluGln CCK-8 have been shown to be stable with long-acting biological effectiveness in genetic and diet-induced obesity-diabetes. Further modification by addition of fatty acid side chain or, as demonstrated here, by PEGylation provides the opportunity to further improve attractiveness of the approach by increasing biological durability. This approach may yield a long-acting form for once or twice-weekly injection. These attributes together with the small size of the molecule, which may facilitate trans-cutaneous administration, make peptidergic CCK-8 analogues a particularly attractive means of harnessing the therapeutic power of the CCK receptor for treatment of obesity, metabolic syndrome, glucose intolerance and obesity.

### FIGURE LEGENDS

FIG. 1 HPLC profiles of CCK-8 and Asp¹-glucitol CCK-8 following incubation with serum for 0, 1 and 2 h on a Vydac C-18 column. Representative traces are shown for CCK-8 (left panels) and Asp¹-glucitol CCK-8 (right panels). Asp¹-glucitol CCK-8 and CCK-8 incubations were separated using linear gradients 0% to 31.5% acetonitrile over 15 min followed by 31.5% to 38.5% over 30 min and 38.5% to 70% acetonitrile over 5 min. Peak A corresponds to intact CCK-8; peaks B, C and D to CCK-8 fragments; and peak E to Asp¹-glucitol CCK-8.

FIG. 2 HPLC profiles of pGlu-Gln CCK-8 following incubation with serum for 0 and 2 h on a Vydac C-18 column. Representative traces are shown for pGlu-Gln CCK-8 after 0 h (left panel) and 2 h (right panel). pGlu-Gln CCK-8 incubations were separated using linear gradients 0% to 31.5% acetonitrile over 15 min followed by 31.5% to 38.5% over 30 min and 38.5% to 70% acetonitrile over 5 min. The eluting single peak at 0 and 2 h corresponds to intact pGlu-Gln CCK-8.

FIG. 3 Effect of CCK-8, Asp¹-glucitol CCK-8, pGlu-Gln CCK-8 or saline on voluntary food intake in Swiss TO mice. Saline or test agents were administered by i.p. injection (100 nmol/kg) to fasted mice at time 0 immediately before introduction of food. Cumulative food intake was monitored at 30, 60, 90, 120, 150 and 180 min post injection. Values are means±SE of 7-8 observations (n=16 for saline controls). Significant differences are indicated by *P<0.05, **P<0.01, ***P<0.001 compared with saline at the same time and ΔP<0.05, ΔΔP<0.01 compared with native CCK-8.

FIG. 4 Effect of CCK-8, Asp¹-glucitol CCK-8 or saline on voluntary food intake in obese diabetic (ob/ob) mice. Saline or test agents were administered by i.p. injection (100 nmol/kg) to fasted obese diabetic (ob/ob) mice at time 0 immediately before introduction of food. Cumulative food intake was monitored at 30, 60, 90, 120, 150, 180, 210, 240, 270 and 300 min post injection. Values are means±SE of 8 observations. Significant differences are indicated by *P<0.05, **P<0.01, ***P<0.001 compared with saline at the same time and ΔP<0.05, ΔΔΔP<0.01 compared with native CCK-8.

FIG. 5 Effect of different doses of CCK-8 or saline on voluntary food intake in Swiss TO mice. Saline or test agents were administered by i.p. injection (1 to 100 nmol/kg) to fasted mice at time 0 immediately before introduction of food. Cumulative food intake was monitored at 30, 60, 90, 120, 150 and 180 min post injection. Values are means±SE of 7-8 observations (n=16 for saline controls). Significant differences are indicated by *P<0.05, **P<0.01, ***P<0.001 compared with saline at the same time.

FIG. 6 Effect of different doses of Asp¹-glucitol CCK-8 or saline on voluntary food intake in Swiss TO mice. Saline or test agents were administered by i.p. injection (1 to 100 nmol/kg) to fasted mice at time 0 immediately before introduction of food. Cumulative food intake was monitored at 30, 60, 90, 120, 150 and 180 min post injection. Values are means±SE of 7-8 observations (n=16 for saline controls). Significant differences are indicated by *P<0.05, **P<0.01, ***P<0.001 compared with saline at the same time.

FIG. 7 Effect of different doses of pGlu-Gln CCK-8 or saline on voluntary food intake in Swiss TO mice. Saline or test agents were administered by i.p. injection (1 to 100 nmol/kg) to fasted mice at time 0 immediately before introduction of food. Cumulative food intake was monitored at 30, 60, 90, 120, 150 and 180 min post injection. Values are means±SE of 7-8 observations (n=16 for saline controls). Significant differences are indicated by *P<0.05, **P<0.01, ***P<0.001 compared with saline at the same time.

FIG. 8 Effect of CCK-8, leptin, combined CCK-8 and leptin, as well as saline on voluntary food intake in Swiss TO mice. Saline or test agents were administered alone (100 nmol/kg) or combined (100 nmol/kg of each) by i.p. injection to fasted mice at time 0 immediately before introduction of food. Cumulative food intake was monitored at 30, 60, 90, 120, 150 and 180 min post injection. Values are means±SE of 7-8 observations. Significant differences are indicated by **P<0.01 compared with saline and **P<0.01 compared to leptin alone at the same time.

FIG. 9 Effect of CCK-8, IAPP, combined CCK-8 and IAPP, as well as saline on voluntary food intake in Swiss TO mice. Saline or test agents were administered alone (100 nmol/kg) or combined (100 nmol/kg of each) by i.p. injection to fasted mice at time 0 immediately before introduction of food. Cumulative food intake was monitored at 30, 60, 90, 120, 150 and 180 min post injection. Values are means±SE of 7-8 observations. Significant differences are indicated by **P<0.01 compared with saline and ΔΔP<0.01 compared to IAPP alone at the same time.

FIG. 10 Effect of pGlu-Gln CCK-8, bombesin, as well as saline on voluntary food intake in Swiss TO mice. Saline or test agents were administered alone (100 nmol/kg) or combined (100 nmol/kg of each) by i.p. injection to fasted mice at time 0 immediately before introduction of food. Cumulative food intake was monitored at 30, 60, 90, 120, 150 and 180 min post injection. Values are means±SE of 7-8 observations. Significant differences are indicated by **P<0.01 compared with saline and ΔΔP<0.01 compared to IAPP alone at the same time.

FIG. 11 Effect of CCK-8, exendin(1-39), combined CCK-8 and exendin(1-39), as well as saline on voluntary food intake in Swiss TO mice. Saline or test agents were administered alone (50 and 100 mmol/kg, respectively) or combined by i.p. injection to fasted mice at time 0 immediately before introduction of food. Cumulative food intake was monitored at 30, 60, 90, 120, 150 and 180 min post injection. Values are means±SE of 7-9 observations. Significant differences are indicated by *P<0.05**P<0.01 ***P<0.001 compared with saline and ΔP<0.05ΔΔP<0.01 ΔΔΔP<0.001 compared to exendin(1-3.9) alone at the same time.

FIG. 12 Effect of pGlu-Gln CCK-8, leptin, combined pGlu-Gln CCK-8 and leptin, as well as saline on voluntary food intake in Swiss TO mice. Saline or test agents were administered alone (pGlu-Gln CCK-8 50 mmol/kg; leptin 100 nmol/kg) or combined by i.p. injection to fasted mice at time 0 immediately before introduction of food. Cumulative food intake was monitored at 30, 60, 90, 120, 150 and 180 min post injection. Values are means±SE of 7-8 observations. Significant differences are indicated by *P<0.05**P<0.01***P<0.001 compared with saline and ΔP<0.05 ΔΔ21 0.01 ΔΔΔP<0.001 compared to leptin alone at the same time.

FIG. 13 illustrates the extensive degradation of CCK-8 to N-terminally truncated forms when incubated with mouse plasma for 120 min. Fragment peptides were separated by reversed-phase HPLC and molecular masses identified by quadripole time of flight (Q-TOF) mass spectrometry. CCK-8, CCK-7, CCK-6 and CCK-5 are indicated by the arrows.

FIG. 14 illustrates lack of degradation of N—Ac—CCK-8 when incubated with mouse plasma for 120 min. HPLC trace shows the elution profile of N—Ac—CCK-8 at time 0 (top panel) and after 120 min (lower panel) exposure to mouse plasma. Reaction mixtures were separated on a Vydac C-18 analytical column (250×4.6 mm). No degradation products of N—Ac—CCK-8 were observed.

FIG. 15 illustrates the protracted dose-dependent inhibitory effects of N—Ac—CCK-8 on feeding in normal mice. N—Ac—CCK-8 (1-100 nmol/kg) or saline (control) was administered by intraperitoneal injection to habituated mice. Food intake was monitored at 30 min intervals up to 180 min. Data are mean±SEM (n=8) of accumulated food intake. *P<0.05, **P<0.01, ***P<0.001 versus saline control.

FIG. 16 illustrates the inhibitory effects of pGluGln-CCK-8 on feeding activity in ob/ob mice on days 1 and 7 of daily dosing. PGluGln-CCK-8 (25 nmol/kg) or saline (control) was administered daily by intraperitoneal injection to adult ob/ob mice for 7 days. Food intake was monitored at intervals immediately after injection on day 1 and day 7. Data are mean±SEM (n=8). PGluGln-CCK-8 was significantly different from saline at all time points (P<0.001).

FIG. 17 illustrates body weight reduction in high fat fed obese mice treated daily with pGluGln-CCK-8. Obese high fat fed mice on reversed light cycle were given twice daily intraperitoneal injections of pGluGlnCCK-8 (25 nmol/kg) or saline at 09.30 and 15.30 h for up to 34 days. Data are mean±SEM (n=8). *P<0.05, **P<0.01, ***P<0.001 compared with saline control.

FIG. 18 illustrates decrease of non-fasting glucose concentrations at 09.00-21.00 h in high fat fed obese mice treated daily with pGluGln-CCK-8. Obese high fat fed mice on reversed light cycle were given twice daily intraperitoneal injections of pGluGlnCCK-8 (25 nmol/kg) or saline at 09.30 and 15.30 h. Blood samples were taken from non-fasted mice on day 32 at times indicated. Data are mean±SEM (n=8). *P<0.05 compared with saline control.

FIG. 19 illustrates lower glycaemic excursion following feeding in high fat fed obese mice treated daily with pGluGln-CCK-8. Obese high fat fed mice on reversed light cycle were given twice daily intraperitoneal injections of pGluGlnCCK-8 (25 nmol/kg) or saline at 09.30 and 15.30 h. Effects of 15 min feeding in overnight fasted mice were examined on day 34. Data are mean±SEM (n=8). **P<0.01, compared with saline control.

FIG. 20 illustrates improved glucose tolerance in high fat fed obese mice treated daily with pGluGln-CCK-8. Obese high fat fed mice on reversed light cycle were given twice daily intraperitoneal injections of pGluGlnCCK-8 (25 nmol/kg) or saline at 09.30 and 15.30 h. Glucose tolerance tests (18 mmol/kg, ip) were conducted on day 34 at 08.30 h. Lower panel shows AUC values for glucose tolerance over 0-60 min. Data are mean±SEM (n=8). *P<0.05, compared with saline control.

FIG. 21 illustrates enhanced insulin sensitivity in high fat fed obese mice treated daily with pGluGln-CCK-8. Obese high fat fed mice on reversed light cycle were given twice daily intraperitoneal injections of pGluGlnCCK-8 (25 nmol/kg) or saline at 09.30 and 15.30 h. Insulin sensitivity tests (20 units/kg, ip) were conducted on day 34 at 08.30 h. Lower panel shows AUC values for glycaemic excursion over 0-60 min. Data are mean±SEM (n=8). *P<0.05, compared with saline control.

FIG. 22 illustrates body weight reduction in high fat fed obese mice treated daily with pGluGln-CCK-8 or pGluGln-CCK-8-Peg. Obese high fat fed mice on reversed light cycle were given twice daily intraperitoneal injections of pGluGlnCCK-8, pGluGln-CCK-8-Peg (both at 25 nmol/kg) or saline at 09.30 and 15.30 h. Data are mean±SEM (n=8). *P<0.05, **P<0.01 compared with saline control.

FIG. 23 illustrates inhibition of food intake in high fat fed obese mice treated daily with pGluGln-CCK-8 or pGluGln-CCK-8-Peg. Obese high fat fed mice on reversed light cycle were given twice daily intraperitoneal injections of pGluGlnCCK-8, pGluGln-CCK-8-Peg (both at 25 nmol/kg) or saline at 09.30 and 15.30 h. Data are mean±SEM (n=8). *P<0.05, **P<0.01, ***P<0.001 compared with saline control.

FIG. 24 illustrates improvement of intraperitoneal glucose tolerance in high fat fed obese mice treated daily with pGluGln-CCK-8 or pGluGln-CCK-8-Peg. Obese high fat fed mice on reversed light cycle were given twice daily intraperitoneal injections of pGluGlnCCK-8, pGluGln-CCK-8-Peg (both at 25 nmol/kg) or saline at 09.30 and 15.30 h. Glucose tolerance tests (18 mmol/kg, ip) were conducted on day 24 at 08.30 h. Data are mean±SEM (n=8). *P<0.05 compared with saline control.

FIG. 25 illustrates the improvement of oral glucose tolerance in high fat fed obese mice treated daily with pGluGln-CCK-8 or pGluGln-CCK-8-Peg. Obese high fat fed mice on reversed light cycle were given twice daily intraperitoneal injections of pGluGlnCCK-8, pGluGln-CCK-8-Peg (both at 25 nmol/kg) or saline at 09.30 and 15.30 h. Oral glucose tolerance tests (18 mmol/kg) were conducted on day 24 at 08.30 h. Responses of lean controls are shown for comparison. Data expressed as change in glucose are mean±SEM (n=8). *P<0.05, **P<0.01 compared with saline control.

FIG. 26 illustrates improved insulin sensitivity in high fat fed obese mice treated daily with pGluGln-CCK-8 or pGluGln-CCK-8-Peg. Obese high fat fed mice on reversed light cycle were given twice daily intraperitoneal injection of pGluGlnCCK-8, pGluGln-CCK-8-Peg (both at 25 nmol/kg) or saline at 09.30 and 15.30 h. Insulin sensitivity tests (20 units/kg, ip) were conducted on day 24 at 08.30 h. Responses of lean controls are shown for comparison. Data expressed as change in glucose are mean±SEM (n=8). **P<0.01, ***P<0.001 compared with saline control.

FIG. 27 illustrates long-lasting effects of pGluGln-CCK-8 and especially pGluGln-CCK-8-Peg on inhibition of feeding when administered acutely to high fat fed obese mice. CCK-8, pGluGln-CCK-8 or pGluGln-CCK-8-Peg (all at 25 nmol/kg, ip) was administered at time=0 to overnight fasted high fat fed obese mice. Food intake was monitored at 30 min intervals up to 180 min. Data are mean±SEM (n=8) of accumulated food intake per time interval. *P<0.05, **P<0.01, ***P<0.001 compared to saline; ΔP<0.05, ΔΔP<0.01, ΔΔΔP<0.001 when N-terminally modified CCK-8 is compared to native CCK; and finally ∞P<0.05, ∞∞P<0.01, ∞∞∞P<0.001 when pGluGlnCCK-8 is compared to pGluGlnCK-8-Peg.

FIG. 28 illustrates that long-lasting effects of pGluGln-CCK-8 and especially pGLuGln-CCK-8-Peg on inhibition of feeding when administered 18 h previously to high fat fed obese mice. CCK-8, pGluGln-CCK-8 or pGluGln-CCK-8-Peg (all at 25 nmol/kg, ip) were administered at time=minus 18 h to overnight fasted high fat fed obese mice. Food intake was monitored at 30 min intervals up to 180 min. Data are mean±SEM (n=8) of accumulated food intake per time interval. *P<0.05, **P<0.01, ***P<0.001 compared to saline; ΔP<0.05, ΔΔP<0.01, ΔΔΔP<0.001 when N-terminally modified CCK-8 is compared to native CCK; and finally ∞P<0.05, ∞∞P<0.01, ∞∞∞P<0.001 when pGluGlnCCK-8 is compared to pGluGlnCK-8-Peg.

FIG. 29 illustrates ineffectiveness of phosphorylated and non-sulphated, as opposed to the native sulphated, form of CCK-8 as inhibitor of feeding in mice. CCK-8 (natural sulphated form), non-sulphated CCK-8, phosphorylated CCK-8 (each at 100 nmol/kg, ip) or saline (control) was administered by intraperitoneal injection to habituated Swiss TO mice. Food intake was monitored at 30 min intervals up to 180 min. Data are mean±SEM (n=8) of accumulated food intake per time interval. *P<0.05,***P<0.001 versus saline; ΔP<0.05, ΔΔΔP<0.001 compared with phosphorylated CCK-8; ++P<0.01, +++P<0.001 compared with non-sulphated CCK-8.

FIG. 30 illustrates powerful stimulatory effects of phosphorylated CCK-8 and pGluGln-CCK-8 on insulin secretion from the clonal pancreatic beta cell line, BRIN-BD11. Effects of native CCK-8, phosphorylated CCK-8 and pGluGln-CCK-8 on insulin release were examined at 5.6 mmol/l glucose. Data are mean±SEM (n=8). *P<0.05, **P<0.01 and ***P<0.001 compared to 5.6 mmol/l glucose alone.

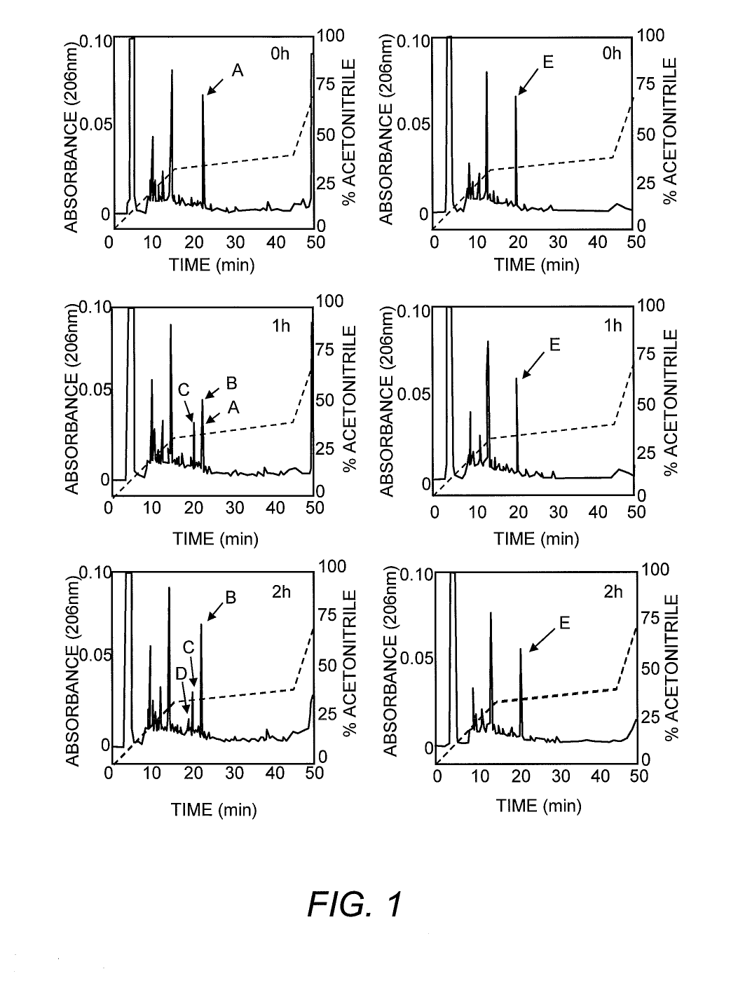

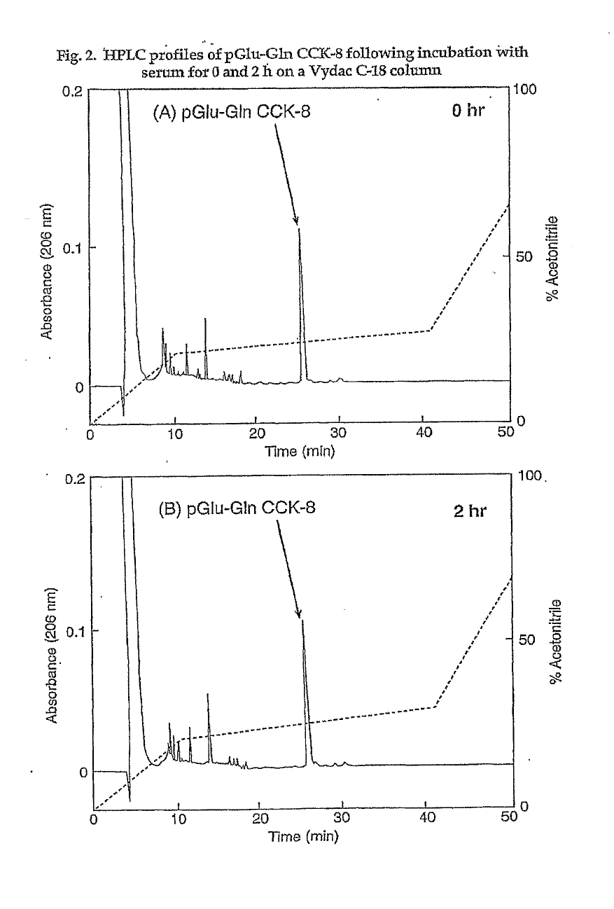

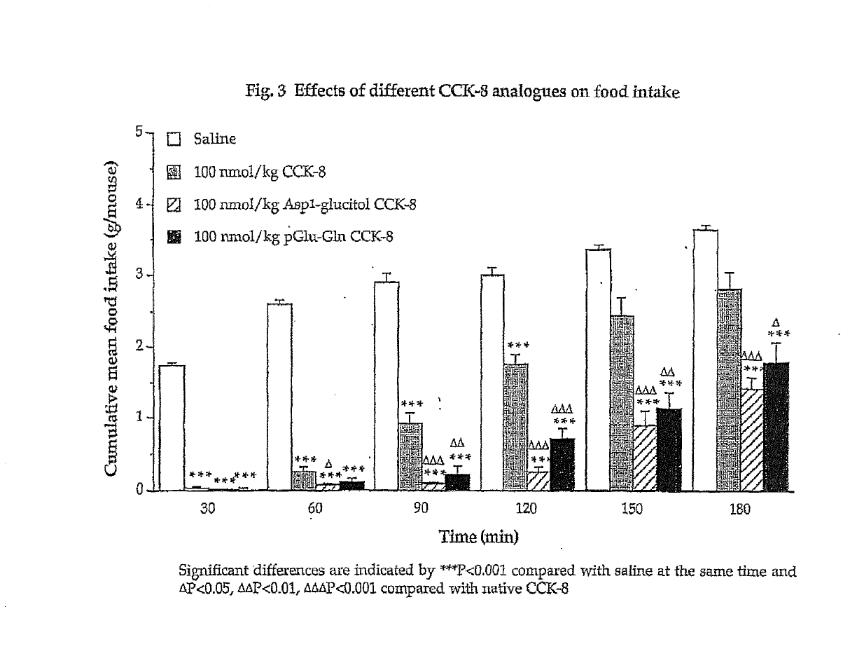

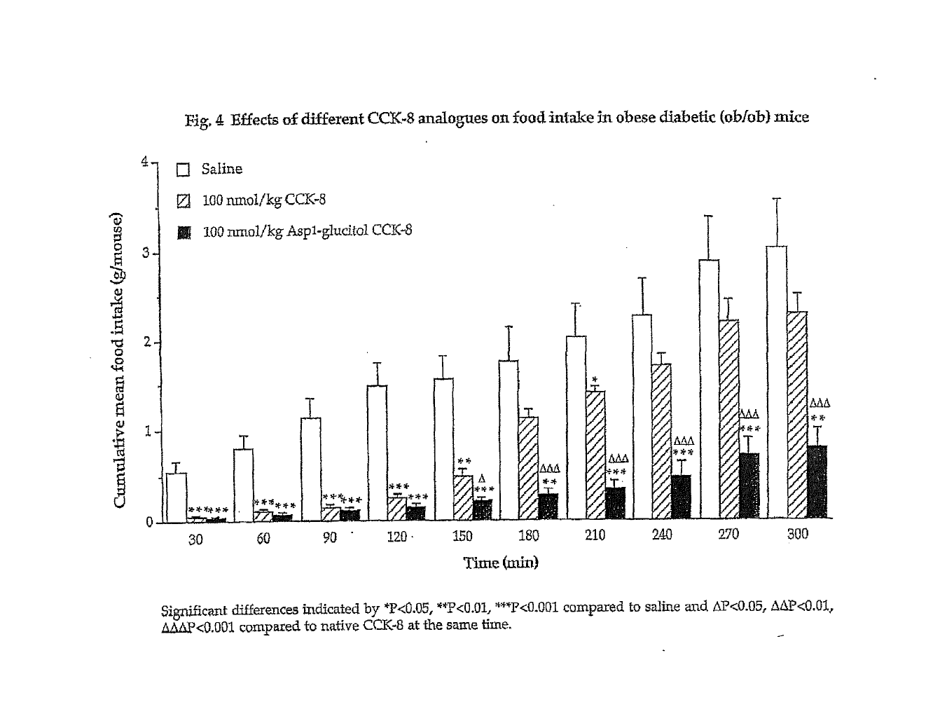

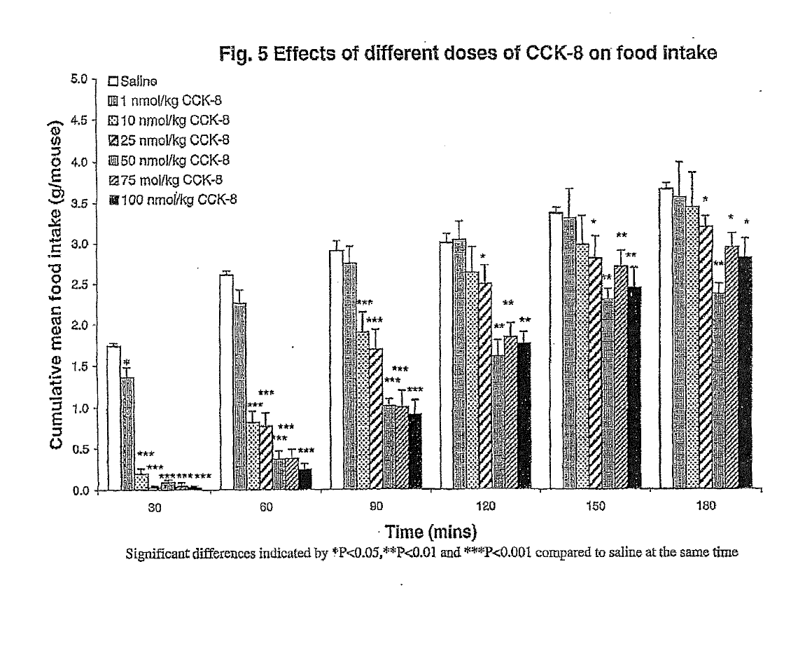

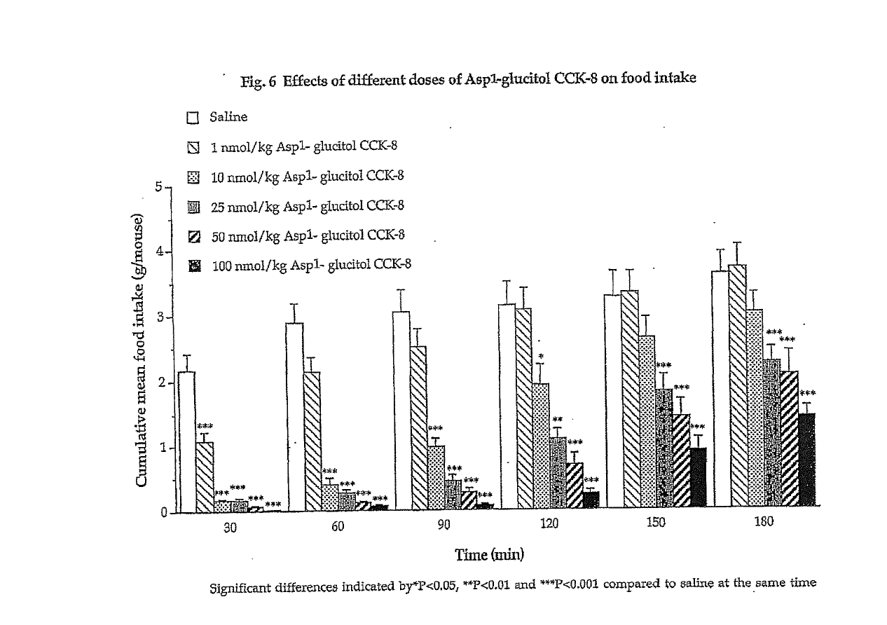

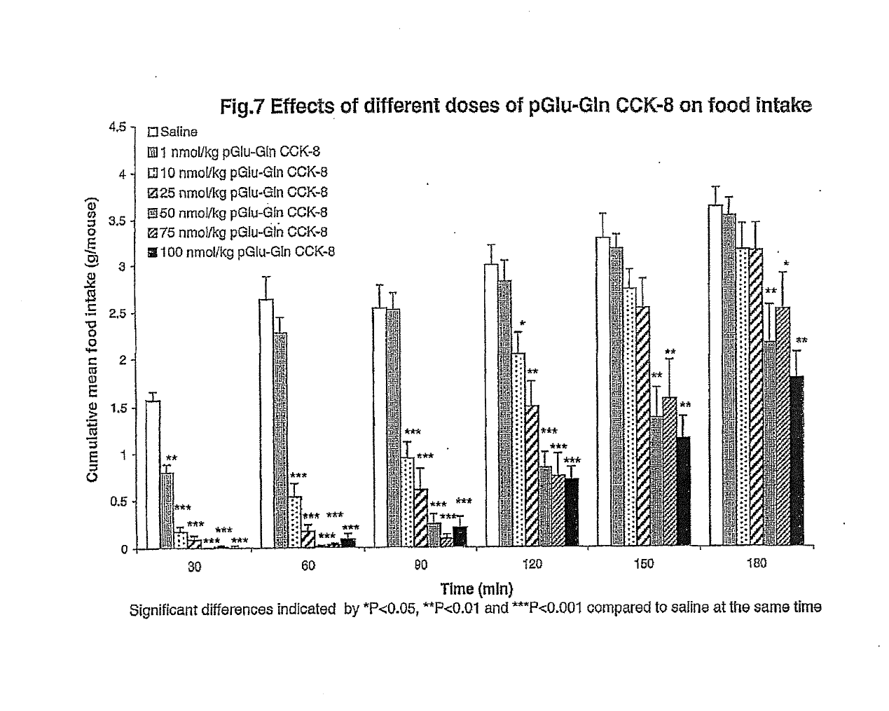

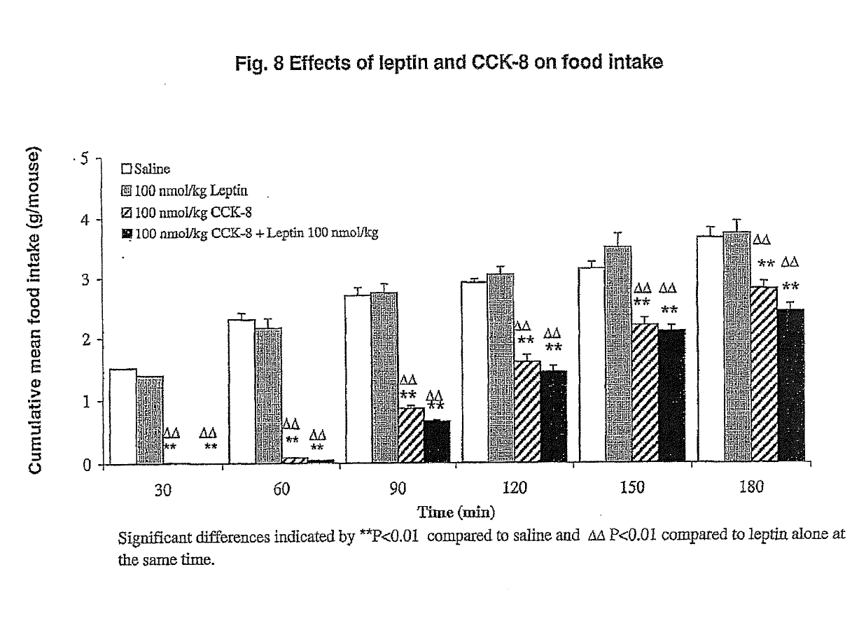

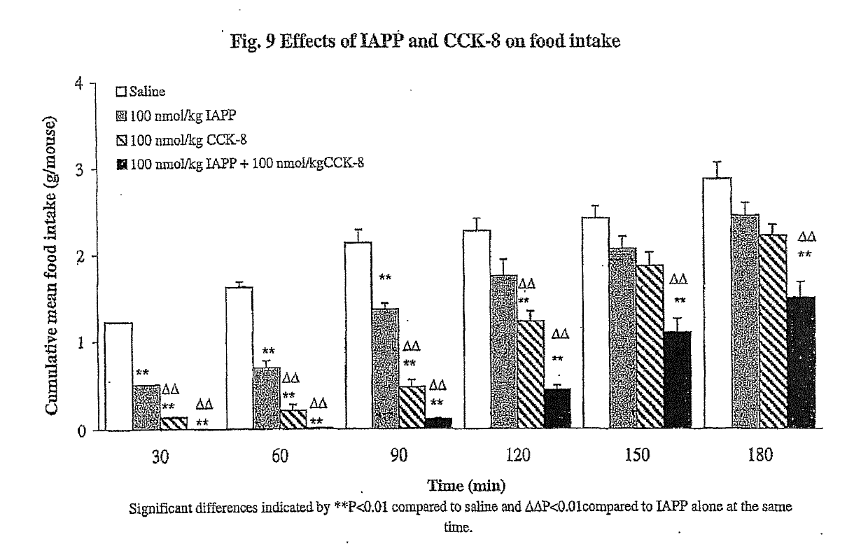

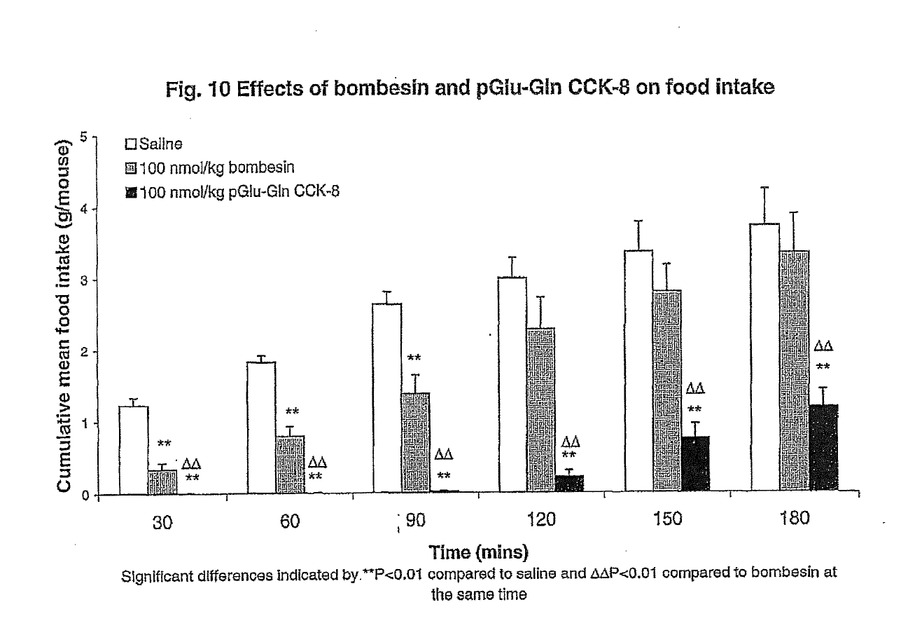

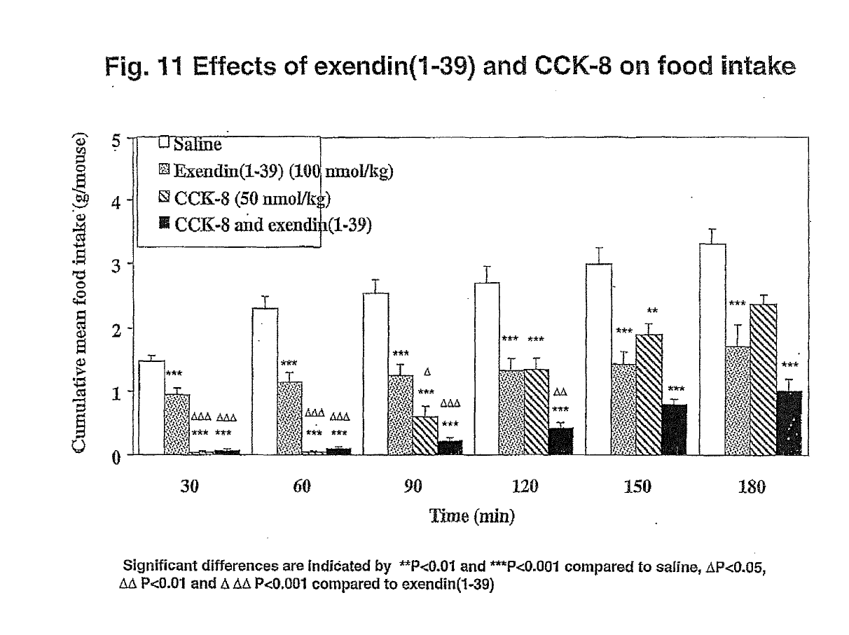

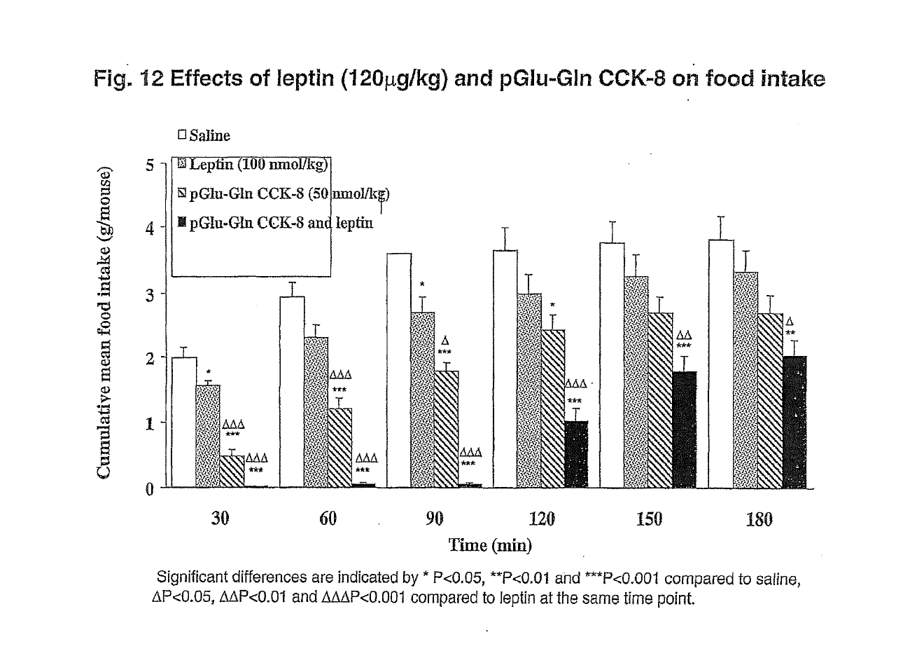

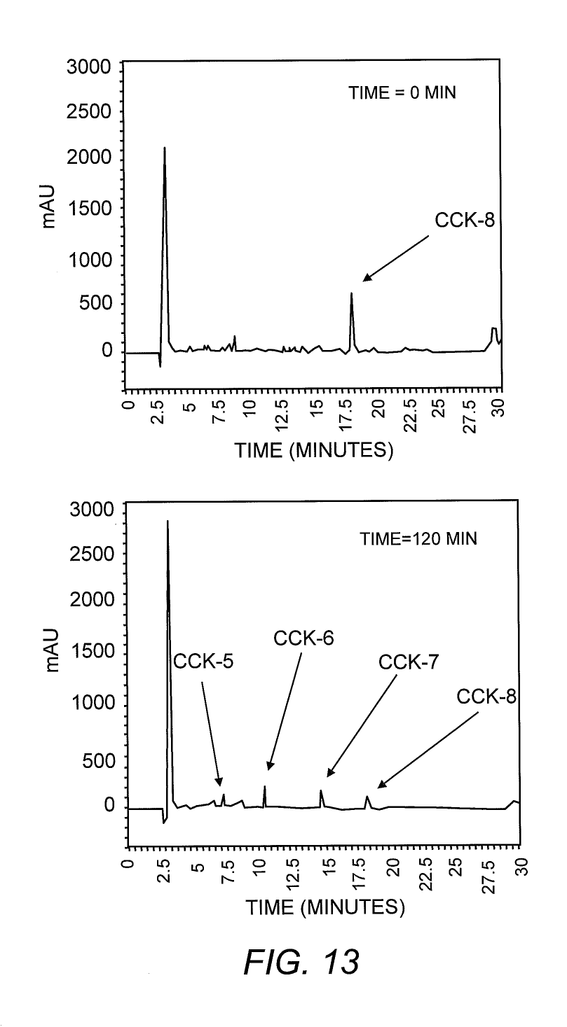

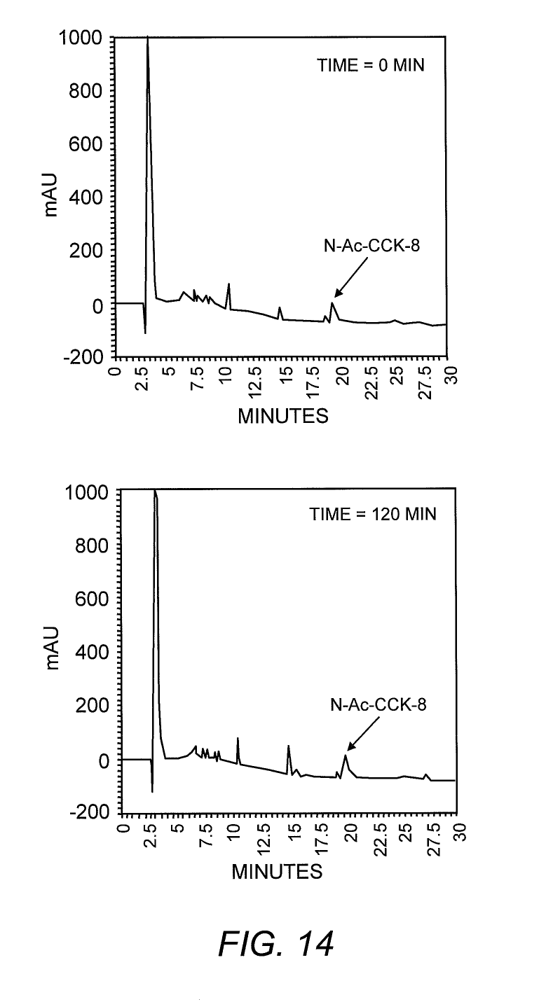

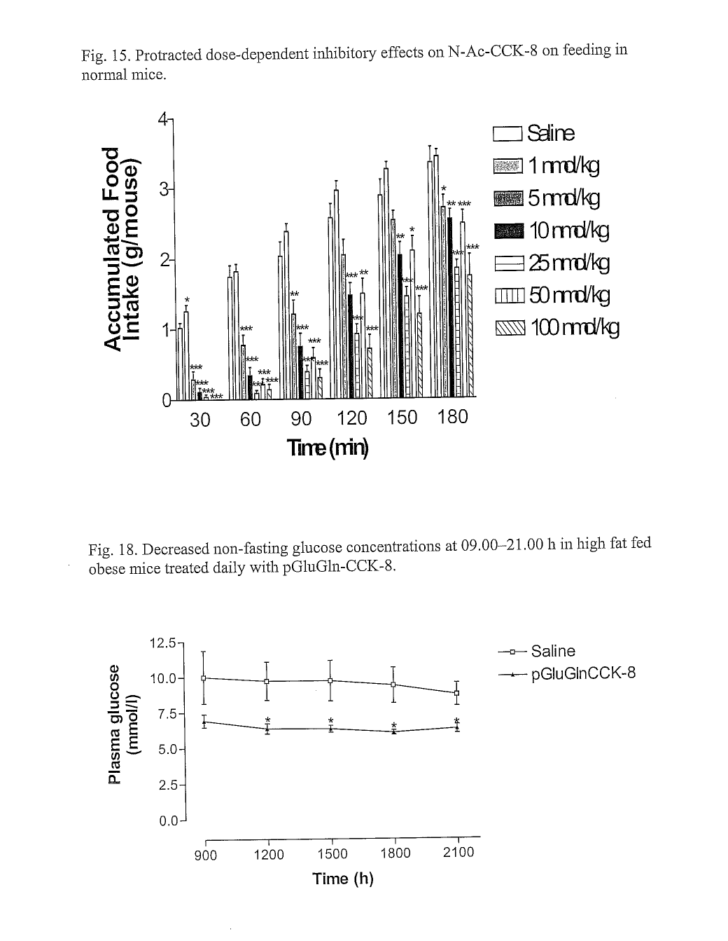

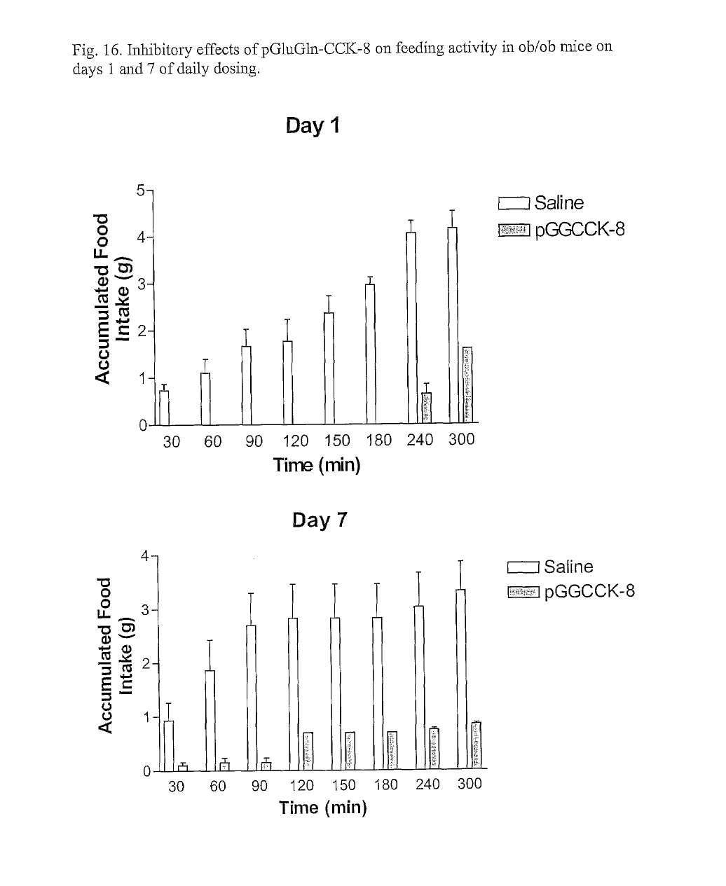

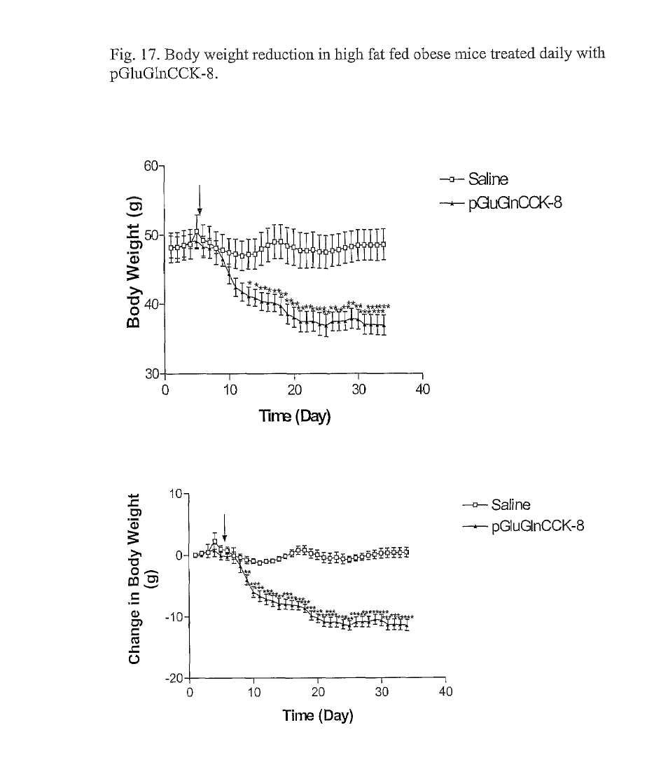

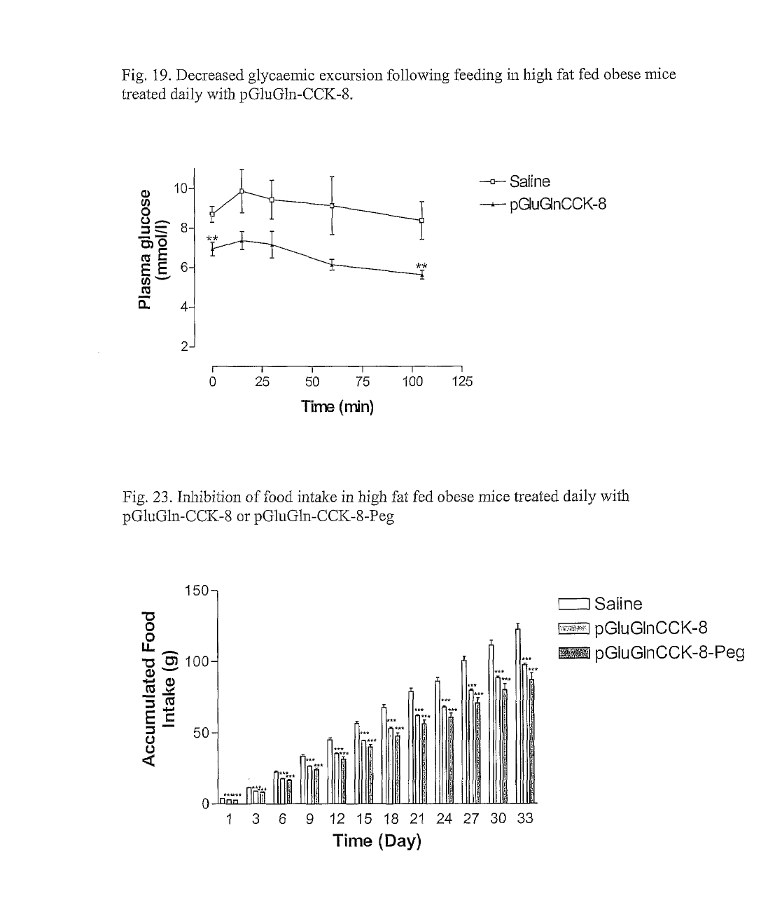

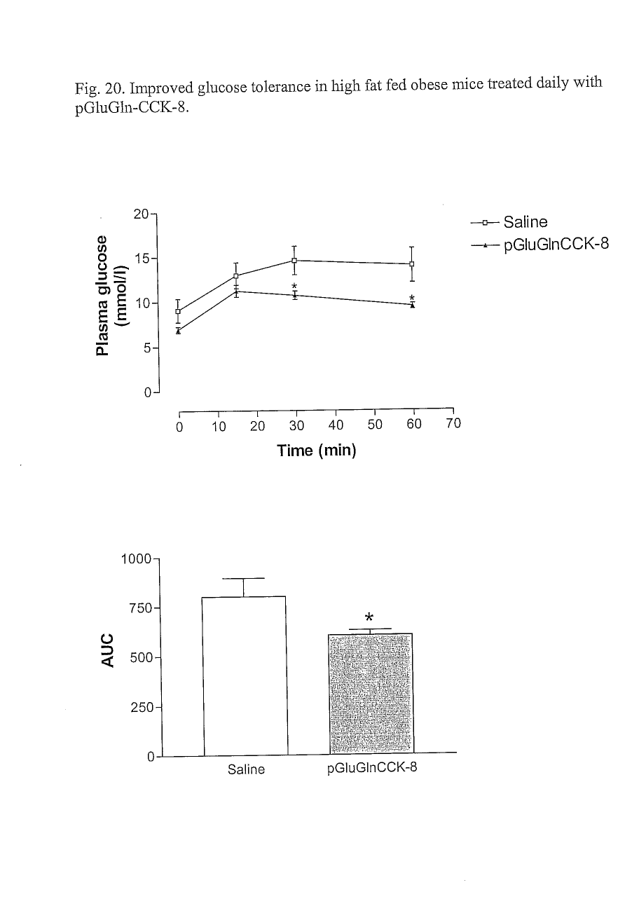

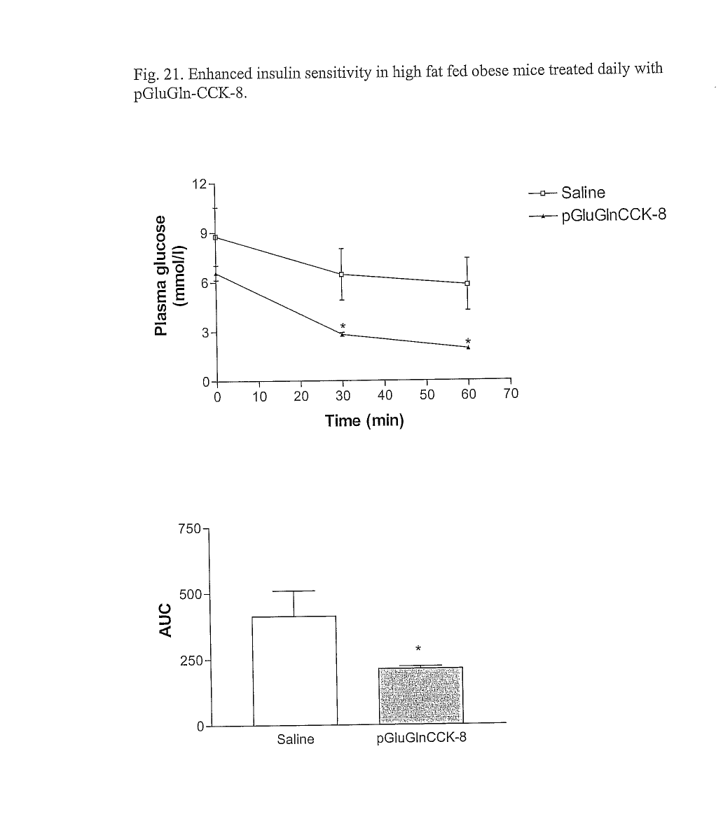

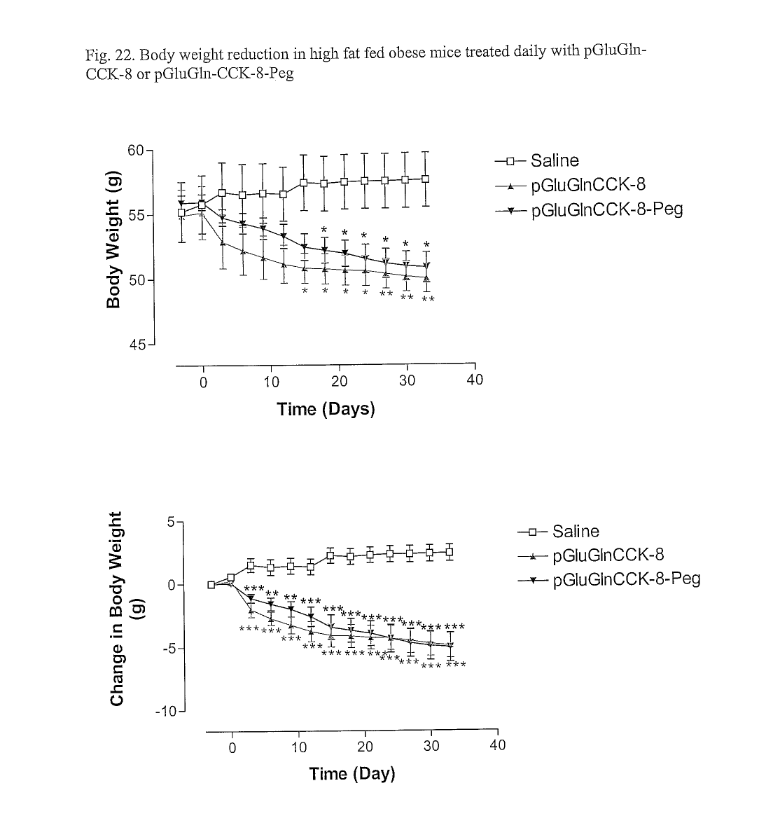

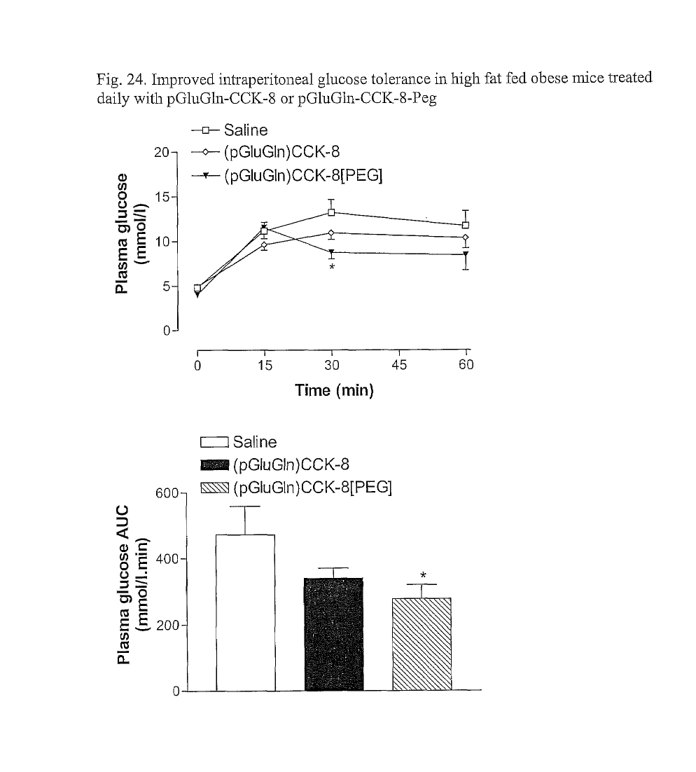

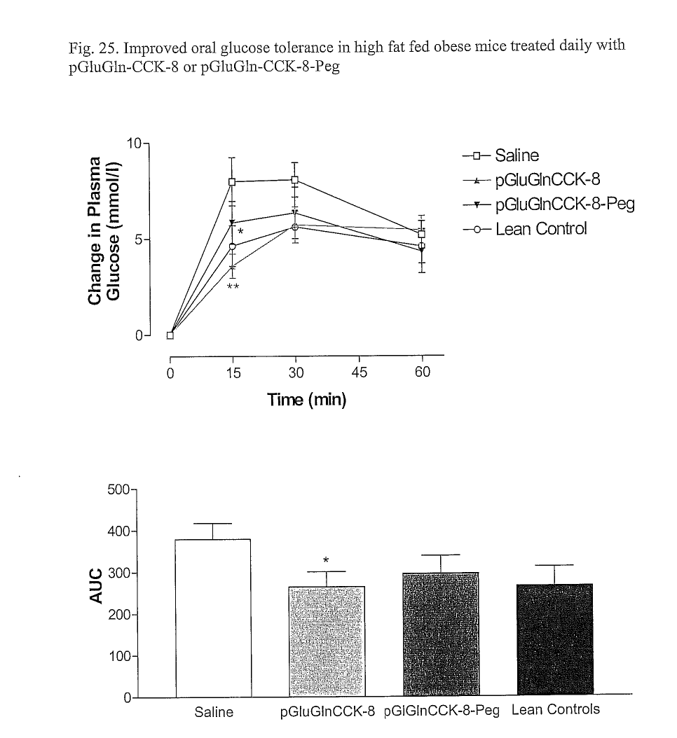

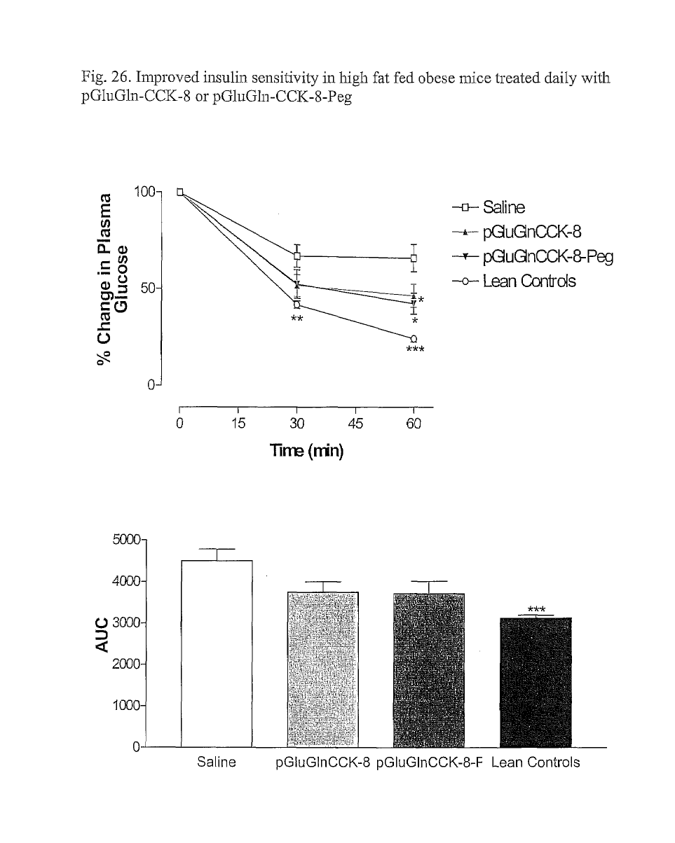

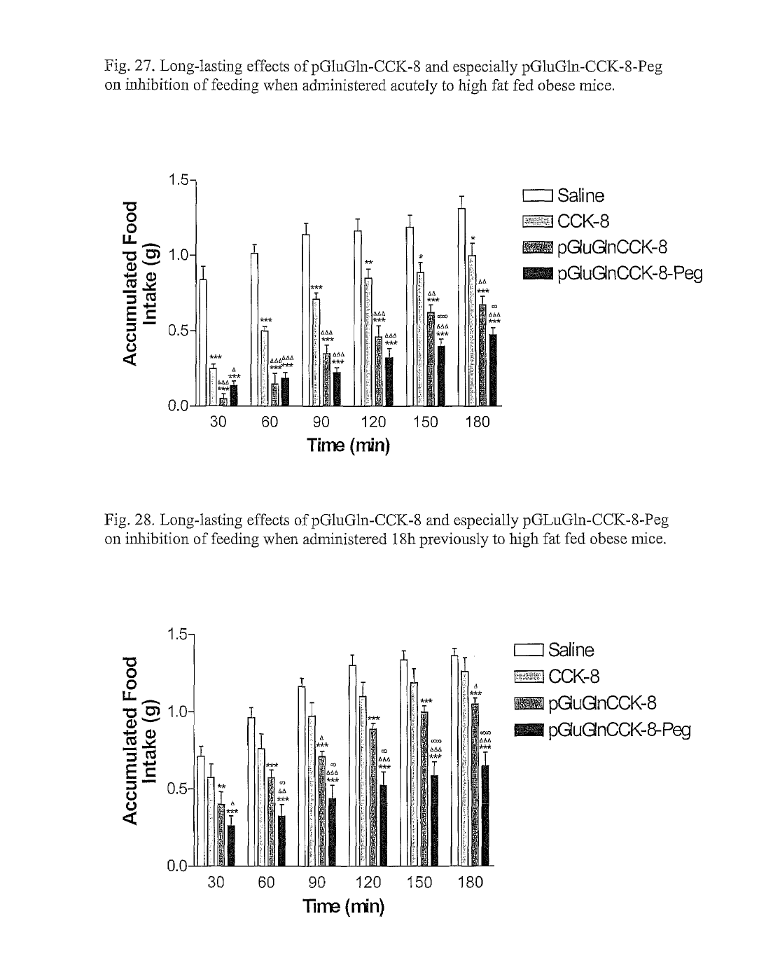

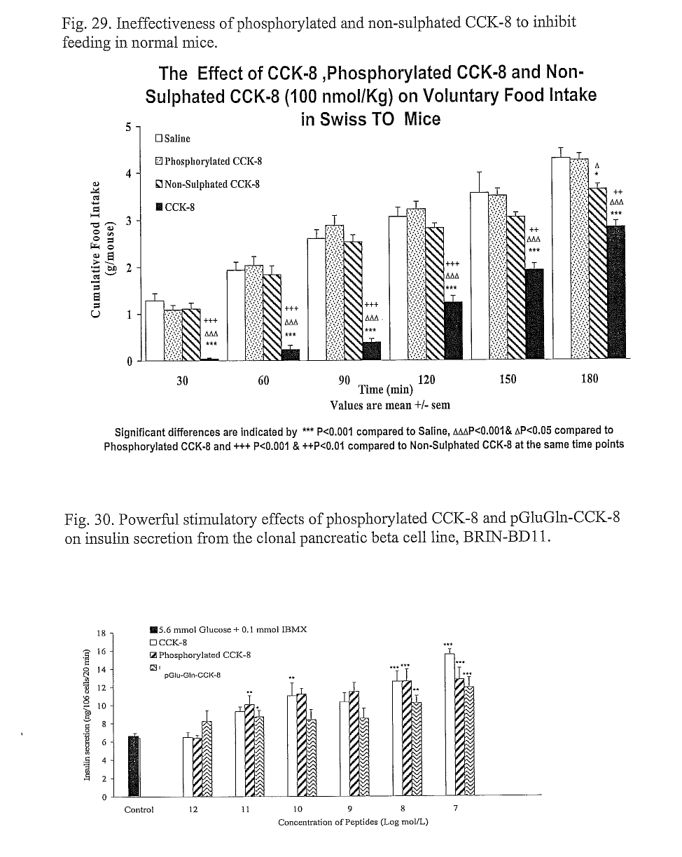

### REFERENCES

Cantor P 1989 Cholecystokinin in plasma. Digestion 42 181-201.
Crawley J N & Corwin R L 1995 Biological actions of cholecystokinin. Peptides 15 731-755.
Gibbs J, Young R C & Smith G P 1973 Cholecystokinin decreases food intake in rats. Journal of comparative and physiological physcology 84 488-495.
Morley J E 1987 Neuropeptide regulation of appetite and weight. Endocrinology reviews 8 256-287.
Innis R B & Synder S H 1980 Distinct cholecystokinin receptors in brain and pancreas. Proceedings of The National Academy of Science USA 77 6917-6921.
Innui A 2000 Transgenic approach to the study of body weight regulation. Pharmacological Reviews 52 33-62.
Liddle R A 1994 Cholecystokinin. In Gut Peptides Biochemistry and Physiology pp 175-216. Eds Walsh J H & Dockray G J. New York: Raven Press.
McClenaghan N H, Barnett C R, Ah-Sing E, Abdel-Wahab Y H A, O'Harte F P M, Yoon T-W, Swanston-Flatt S K & Flatt P R 1996 Characterization of a novel glucose-responsive insulin-secreting cell line, BRIN-BD11, produced by electrofusion. Diabetes 45: 1132-1140.
Silver A J & Morley J E 1991 Role of CCK in the regulation of food intake. Progress in Neurobiology 36 23-34.
Smith G P 1984 The therapeutic potential of cholecystokinin. International Journal of Obesity 8 35-38.
Ukkola O 2004 Peripheral regulation of food intake: new insights. Journal of Endocrinolgy Investigations 27 96-98.
Wynne K, Stanley S, Mcgowan B & Bloom S 2005 Appetite control. Journal of Endocrinology 184 291-318

The invention is not limited to the embodiment(s) described herein but can be amended or modified without departing from the scope of the present invention.

1. A method of N-terminally modifying CCK-8 and analogues thereof comprising the steps of solid phase synthesis of the C-terminus of CCK-8 up to Met³, adding Tyr(tBu) as an Fmoc-protected PAM resin, deprotecting the Fmoc by piperidine in DMF and reacting with an Fmoc protected Asp(OtBu)-OH, allowing the reaction to proceed to completion, removing the Fmoc protecting group from the dipeptide, reacting the dipeptide with a modifying agent, removing side-chain protecting groups (tBu and OtBu) by acid, sulphating the Tyr² with sulphur trioxide, and cleaving the N-terminal modified dipeptide from the resin under alkaline conditions.

2. A method as claimed in claim 1 further including the step of adding the N-terminal modified dipeptide to the C-terminal peptide resin in the synthesizer, followed by cleavage from the resin under alkaline conditions with methanolic ammonia.

3. A peptide based on biologically active CCK-8, the peptide having improved characteristics for the treatment of at least one of obesity and type 2 diabetes, wherein the structure of the peptide is:
(Z)-Asp¹-Aaa²(X)-Aaa³Gly⁴Trp⁵Aaa⁶Asp⁷(Y)Aaa⁸K,
wherein:
the amino acids may be either D or L amino acids;
the bond between amino acid residues is either a peptide bond or a non-peptide isostere bond;
Aaa² is selected from the group comprising Tyr and Phe;
when Aaa² is Tyr, X is selected from the group comprising SO₃H⁻, PO₃H₂⁻ and a polymer moiety of the general formula —O—(CH₂—O—CH₂)ₙ—H, in which n is an integer between 1 and about 22, wherein the X is covalently bound to the para phenyl oxygen of Tyr, and, when Aaa² is Phe, X is CH₂SO₃Na, wherein the X is covalently bound to the para phenyl position of Phe;
Aaa³ is selected from the group comprising Met, norleucine, 2-aminohexanoic acid and Thr;
Aaa⁶ is selected from the group comprising Met, norleucine, 2-aminohexanoic acid and Phe;
Aaa⁸ is selected from the group comprising Phe and Met;
(Y)Aaa⁸K, when Aaa⁸ is Phe⁸ and K is an amide, is: 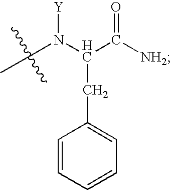
Y is covalently bound to nitrogen and is selected from the group consisting of H and CH₃;
K is selected from the group consisting of the hydroxyl group of Phe⁸, an amide covalently bound to Phe⁸, an ester covalently bound to Phe⁸, a salt of the hydroxyl group of Phe⁸, a salt of an amide covalently bound to Phe⁸, a salt of an ester covalently bound to Phe⁸ and a polymer moiety of the general formula —O—(CH₂—O—CH₂)ₙ—H, in which n is an integer between 1 and about 22; and
Z comprises at least one amino acid modification, wherein said at least one modification comprises an N-terminal extension, or an N-terminal modification, but excludes Asp¹-glucitol CCK-8 where Aaa² is Tyr and X is SO₃H⁻.

4. A peptide as claimed in claim 3 wherein the structure of the peptide is:
(Z)-Asp¹-Aaa²(X)-Aaa³Gly⁴Trp⁵Aaa⁶Asp⁷(Y)Aaa⁸K,
wherein:
the amino acids are L amino acids;
the bonds between amino acid residues are peptide bonds;
Aaa³ and Aaa⁶ are each Met;
Aaa⁸ is Phe;
Aaa²(X) is Tyr²(X) being; 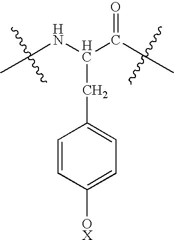
X is covalently bound to oxygen and selected from the group consisting of SO₃H⁻, PO₃H₂⁻ and a polymer moiety of the general formula —O—(CH₂—O—CH₂)ₙ—H, in which n is an integer between 1 and about 22;
K is an amide covalently bound to Phe⁸; and
Y is selected from the group consisting of H and CH₃.

5. A peptide as claimed in claim 3 wherein said N-terminal modification at position 1 is selected from the group comprising N-alkylation, N-acetylation, N-acylation, N-glycation and N-isopropylation of the amino acid at position 1.

6. A peptide as claimed in claim 3 wherein said N-terminal extension is selected from the group comprising pGlu, pGlu-Gln, an acid, a fatty acid, Boc, Fmoc, Arg and attachment of a polymer moiety of the general formula HO—(CH₂—O—CH₂)ₙ—H, in which n is an integer between 1 and about 22.

7. A peptide as claimed in claim 3 further comprising replacement of any amino acid with Lys.

8. A peptide as claimed in claim 7 further comprising fatty acid addition at an epsilon amino group of at least one substituted lysine residue.

9. A peptide as claimed in claim 3 further comprising attachment to Asp⁷ of a polymer moiety of the general formula HO—(CH₂—O—CH₂)ₙ—H, in which n is an integer between 1 and about 22.

10. A peptide as claimed in claim 3 further comprising replacement of any amino acid with an amino acid selected from the group including, but not limited to, lysine, cysteine, histidine, arginine, aspartic acid, glutamic acid, serine, threonine, and tyrosine and attachment of a polymer moiety of the general formula HO—(CH₂—O—CH₂)ₙ—H, in which n is an integer between 1 and about 22 to at least one substituted amino acid.

11. A peptide as claimed in claim 3 wherein Z is selected from the group consisting of:
(i) N-terminal extension of the peptide by pGlu-Gln and Aaa⁸ is Phe;
(ii) N-terminal extension of the peptide by pGlu-Gln and Aaa⁸ is Met;
(iii) N-terminal extension of the peptide by Arg;
(iv) N-terminal extension of the peptide by pyroglutamyl (pGlu);
(v) modification of Asp¹ by acetylation;
(vi) modification of Asp¹ by acylation;
(vii) modification of Asp¹ by alkylation or glycation;
(viii) modification of Asp¹ by isopropylation;
(ix) N-terminal extension of the peptide at Asp¹ by Fmoc or Boc;
(x) N-terminal extension or an N-terminal modification and there are D-amino acid substituted CCK-8 at one or more amino acid sites;
(xi) N-terminal extension of the peptide by attachment of a polymer moiety of the general formula HO—(CH₂—O—CH₂)ₙ—H, in which n is an integer between 1 and about 22; and
(xii) N-terminal extension of the peptide by pGlu-Gln and C-terminal extension of the peptide by attachment of a polymer moiety of the general formula HO—(CH₂—O—CH₂)ₙ—H, in which n is an integer between 1 and about 22.

12. The peptide of claim 3 wherein K comprises a polymer moiety covalently bound to Phe⁸, the polymer moiety being of the general formula —O—(CH₂—O—CH₂)ₙ—H, in which n is an integer between 1 and about 22.

13. The peptide of claim 3, wherein n is an integer between 1 and about 10.

14. The peptide of claim 12, wherein n is an integer between about 2 and about 6.

15. The peptide of claim 12 wherein the peptide is further modified by N-terminal extension of the peptide.

16. The peptide of claim 15 wherein the peptide is modified by N-terminal extension of the peptide by pGlu-Gln.

17. The peptide of claim 3 wherein:
the amino acids are L amino acids;
the bonds between amino acid residues are peptide bonds;
Aaa³ and Aaa⁶ are each Met;
Aaa⁸ is Phe;
Aaa² is Tyr;
X is PO₃H₂⁻;
K is an amide covalently bound to Phe⁸; and
Y is H.

18. The peptide of claim 3 wherein:
the amino acids are L amino acids;
the bonds between amino acid residues are peptide bonds;
Aaa³ and Aaa⁶ are each Met;
Aaa⁸ is Phe;
Aaa² is Tyr;
X is SO₃H⁻;
K is an amide covalently bound to Phe⁸;
Y is H; and
the peptide is modified by N-terminal acetylation of Asp¹.

19. A peptide as claimed in claim 3 wherein at least one peptide isostere bond is present between amino acid residues at any site within the peptide.

20. A peptide as claimed in claim 19 wherein the isostere bond is present between Asp¹-Tyr²; between Tyr²-Met³; between Met³-Gly⁴; or between Met⁶-Asp⁷.

21. A peptide as claimed in claim 11 wherein Z is selected from the group consisting of:
(i) N-terminal extension of the peptide by pGlu-Gln;
(ii) N-terminal extension of the peptide by Arg;
(iii) N-terminal extension of the peptide by pyroglutamyl (pGlu);
(iv) modification of Asp¹ by acetylation;
(v) modification of Asp¹ by acylation;
(vi) modification of Asp¹ by alkylation or glycation; and
(vii) modification of Asp¹ by isopropylation.

22. A fragment of the peptide of claim 3, wherein the structure of the peptide fragment is:
(Z)-Aaa²(X)-Aaa³Gly⁴Trp⁵Aaa⁶Asp⁷(Y)Aaa⁸K,
wherein:
the amino acids may be either D or L amino acids;
the bond between amino acid residues is either a peptide bond or a non-peptide isostere bond;
Aaa² is selected from the group comprising Tyr and Phe;
when Aaa² is Tyr, X is selected from the group comprising SO₃H⁻, PO₃H₂⁻ and a polymer moiety of the general formula —O—(CH₂—O—CH₂)ₙ—H, in which n is an integer between 1 and about 22, wherein the X is covalently bound to the para phenyl oxygen of Tyr, and, when Aaa² is Phe, X is CH₂SO₃Na, wherein the X is covalently bound to the para phenyl position of Phe;
Aaa³ is selected from the group comprising Met, norleucine, 2-aminohexanoic acid and Thr;
Aaa⁶ is selected from the group comprising Met, norleucine, 2-aminohexanoic acid and Phe;
Aaa⁸ is selected from the group comprising Phe and Met;
(Y)Aaa⁸K, when Aaa⁸ is Phe and K is an amide, is: 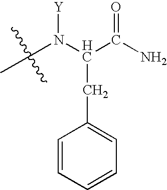
Y is covalently bound to nitrogen and is selected from the group consisting of H and CH₃;
K is selected from the group consisting of the hydroxyl group of Phe⁸, an amide covalently bound to Phe⁸, an ester covalently bound to Phe⁸, a salt of the hydroxyl group of Phe⁸, a salt of an amide covalently bound to Phe⁸, a salt of an ester covalently bound to Phe⁸ and a polymer moiety covalently bound to Phe⁸, the polymer moiety being of the general formula —O—(CH₂—O—CH₂)ₙ—H, in which n is an integer between 1 and about 22; and
Z comprises at least one amino acid modification, wherein said at least one modification comprises an N-terminal extension, or an N-terminal modification.

23. A fragment as claimed in claim 22 wherein the structure of the peptide fragment is:
(Z)-Aaa²(X)-Aaa³Gly⁴Trp⁵Aaa⁶Asp⁷(Y)Aaa⁸K,
wherein:
the amino acids are L amino acids;
the bonds between amino acid residues are peptide bonds;
Aaa³ and Aaa⁶ are each Met;
Aaa⁸ is Phe;
Aaa²(X) is Tyr²(X): 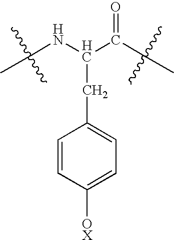
X is covalently bound to oxygen and selected from the group consisting of SO₃H⁻, PO₃H₂⁻ and a polymer moiety of the general formula —O—(CH₂—O—CH₂)ₙ—H, in which n is an integer between 1 and about 22;
K is an amide covalently bound to Phe⁸; and
Y is selected from the group consisting of H and CH₃.

24. A fragment as claimed in claim 22 wherein said N-terminal modification is selected from the group comprising N-alkylation, N-acetylation, N-acylation, N-glycation, or N-isopropylation at Aaa².

25. A fragment as claimed in claim 24, wherein Aaa² is Tyr and said N-terminal modification is selected from the group comprising:
(i) acetylation of Tyr²;
(ii) glycation of Tyr²; and
(iii) acylation of Tyr² by succinic acid.

26. A fragment as claimed in claim 22 wherein said N-terminal extension is selected from the group comprising pGlu, pGlu-Gln, an acid, a fatty acid, Boc, Fmoc, Arg and a polymer moiety of the general formula —O—(CH₂—O—CH₂)ₙ—H, in which n is an integer between 1 and about 22.

27. A fragment as claimed in claim 26, wherein said N-terminal extension is selected from the group comprising:
(i) modification of Tyr² by pyroglutamyl;
(ii) modification of Tyr² by Fmoc; and
(iii) modification of Tyr² by Boc.

28-29. (canceled)

30. A pharmaceutical composition including a peptide as claimed in claim 3.

31. A pharmaceutical composition useful in the treatment of at least one of obesity and type 2 diabetes, which comprises an effective amount of a peptide as claimed in claim 3 in admixture with a pharmaceutically acceptable excipient for delivery through transdermal, nasal inhalation, oral or injected routes.

32. A pharmaceutical composition as claimed in claim 31 which further comprises native or derived analogues of leptin, exendin, islet amyloid polypeptide or bombesin.

33. A method for treating at least one of obesity and type 2 diabetes, the method comprising administering to an individual in need of such treatment an effective amount of a peptide as claimed in claim 3 thereby treating obesity or type 2 diabetes.

34. The peptide of claim 4, wherein n is an integer between 1 and about 10.

35. The peptide of claim 6, wherein n is an integer between 1 and about 10.

36. The peptide of claim 12, wherein n is an integer between 1 and about 10.

37. A method for inhibiting food intake, inducing satiety, stimulating insulin secretion, moderating blood glucose excursions, or enhancing glucose disposal in a subject comprising administering to an individual in need of such treatment an effective amount of a peptide of claim 3 thereby inhibiting food intake, inducing satiety, stimulating insulin secretion, moderating blood glucose excursions, or enhancing glucose disposal in the subject.

38. A method for inhibiting food intake, inducing satiety, stimulating insulin secretion, moderating blood glucose excursions, or enhancing glucose disposal in a subject comprising administering to an individual in need of such treatment an effective amount of a peptide of claim 4 thereby inhibiting food intake, inducing satiety, stimulating insulin secretion, moderating blood glucose excursions, or enhancing glucose disposal in the subject.

39. A method for inhibiting food intake, inducing satiety, stimulating insulin secretion, moderating blood glucose excursions, or enhancing glucose disposal in a subject comprising administering to an individual in need of such treatment an effective amount of a fragment of claim 22 thereby inhibiting food intake, inducing satiety, stimulating insulin secretion, moderating blood glucose excursions, or enhancing glucose disposal in the subject.

40. A method for inhibiting food intake, inducing satiety, stimulating insulin secretion, moderating blood glucose excursions, or enhancing glucose disposal in a subject comprising administering to an individual in need of such treatment an effective amount of a fragment of claim 23 thereby inhibiting food intake, inducing satiety, stimulating insulin secretion, moderating blood glucose excursions, or enhancing glucose disposal in the subject.

41. A method for treating at least one of obesity and type 2 diabetes, the method comprising administering to an individual in need of such treatment an effective amount of a peptide of claim 4 thereby treating obesity or type 2 diabetes.

42. A method for treating at least one of obesity and type 2 diabetes, the method comprising administering to an individual in need of such treatment an effective amount of a fragment of claim 22 thereby treating obesity or type 2 diabetes

43. A method for treating at least one of obesity and type 2 diabetes, the method comprising administering to an individual in need of such treatment an effective amount of a fragment of claim 23 thereby treating obesity or type 2 diabetes.

44. A pharmaceutical composition including a peptide of claim 4.

45. A pharmaceutical composition including a fragment of claim 22.

46. A pharmaceutical composition including a fragment of claim 23.

47. A pharmaceutical composition useful in the treatment of at least one of obesity and type 2 diabetes, which comprises an effective amount of a peptide of claim 4 in admixture with a pharmaceutically acceptable excipient for delivery through transdermal, nasal inhalation, oral or injected routes.

48. A pharmaceutical composition useful in the treatment of at least one of obesity and type 2 diabetes, which comprises an effective amount of a fragment of claim 22 in admixture with a pharmaceutically acceptable excipient for delivery through transdermal, nasal inhalation, oral or injected routes.

49. A pharmaceutical composition useful in the treatment of at least one of obesity and type 2 diabetes, which comprises an effective amount of a fragment of claim 23 in admixture with a pharmaceutically acceptable excipient for delivery through transdermal, nasal inhalation, oral or injected routes.
